SEQUENCE LISTING (27 sequences)

SEQ ID NO: 1 - RNA, 26 nt, Artificial Sequence
  oligonucleotide
ggnnnnnnnnnnnnnnnnnnnnnngg

SEQ ID NO: 2 - RNA, 26 nt, Artificial Sequence
  oligonucleotide
cccccccccccccccccccccccccc

SEQ ID NO: 3 - DNA, 23 bp, Artificial Sequence
  oligonucleotide
nnnnnnnnnnnnnnngtgatatg

SEQ ID NO: 4 - DNA, 23 bp, Artificial Sequence
  oligonucleotide
ccccccccccccccccatatcac

SEQ ID NO: 5 - DNA, 23 bp, Artificial Sequence
  oligonucleotide
gnnnnnnnnnnnnnnngtgatat

SEQ ID NO: 6 - DNA, 23 bp, Artificial Sequence
  oligonucleotide
cccccccccccccccccatauca

SEQ ID NO: 7 - DNA, 22 bp, Artificial Sequence
  oligonucleotide
tgnnnnnnnnnnnnnnnttata

SEQ ID NO: 8 - DNA, 23 bp, Artificial Sequence
  oligonucleotide
acccccccccccccccccatatc

SEQ ID NO: 9 - RNA, 32 nt, Artificial Sequence
  oligonucleotide
ggnnnnnnnnnnnnnnnaaaaaaaaaaaaaaa

SEQ ID NO: 10 - RNA, 32 nt, Artificial Sequence
  oligonucleotide
cccccccccccccccccuuuuuuuuuuuuuuu

SEQ ID NO: 11 - RNA, 22 nt, Artificial Sequence
  oligonucleotide
ggnnnnnnnnnnnaaaaaaaaa

SEQ ID NO: 12 - RNA, 22 nt, Artificial Sequence
  oligonucleotide
uuuuuuuuuucccccccccccc

SEQ ID NO: 13 - DNA, 40 bp, Artificial Sequence
  oligonucleotide
ggnnnnnnnnnnnnnnngtcgttnnnnnnnnnnnnnnngg

SEQ ID NO: 14 - RNA, 40 nt, Artificial Sequence
  oligonucleotide
cccccccccccccccccaacgacccccccccccccccccc

SEQ ID NO: 15 - DNA, 23 bp, Artificial Sequence
  oligonucleotide
ggnnnnnnnnnnnnnnngtcgtt

SEQ ID NO: 16 - RNA, 23 nt, Artificial Sequence
  oligonucleotide
aacgacccccccccccccccccc

SEQ ID NO: 17 - DNA, 21 bp, Artificial Sequence
  oligonucleotide
nnnnnnnnnnnnnnngtcgtt

SEQ ID NO: 18 - RNA, 21 nt, Artificial Sequence
  oligonucleotide
cccccccccccccccaacgac

SEQ ID NO: 19 - DNA, 22 bp, Artificial Sequence
  oligonucleotide
gnnnnnnnnnnnnnnngtcgtt

SEQ ID NO: 20 - RNA, 22 nt, Artificial Sequence
  oligonucleotide
ccccccccccccccccaacgac

SEQ ID NO: 21 - DNA, 23 bp, Artificial Sequence
  oligonucleotide
cgnnnnnnnnnnnnnnngtcgtt

SEQ ID NO: 22 - RNA, 23 nt, Artificial Sequence
  oligonucleotide
gccccccccccccccccaacgac

SEQ ID NO: 23 - DNA, 6 bp, Artificial Sequence
  oligonucleotide
gtcgtt

SEQ ID NO: 24 - RNA, 6 nt, Artificial Sequence
  oligonucleotide
aacgac

SEQ ID NO: 25 - RNA, 22 nt, Artificial Sequence
  oligonucleotide
ccccccccccccuuuuuuuuuu

SEQ ID NO: 26 - DNA, 33 bp, Artificial Sequence
  oligonucleotide
ggcgtcgttaaaaaaaaaaaaaaaatgtcgttg

SEQ ID NO: 27 - DNA, 33 bp, Artificial Sequence
  oligonucleotide
uuuuuuuuuuuuuuutaacgacgcccaacgaca

(19) United States
(12) Patent Application Publication
(10) Pub. No.: US 2009/0041809 A1
(43) Pub. Date: Feb. 12, 2009

(54) DOUBLE-STRANDED LOCKED NUCLEIC ACID COMPOSITIONS

(75) Inventor: Peter Emtage, San Diego, CA (US)

Correspondence Address: SUGHRUE MION, PLLC, 2100 PENNSYLVANIA AVENUE, N.W., SUITE 800, WASHINGTON, DC, 20037, US

(73) Assignee: NVENTA BIOPHARMACEUTICALS CORPORATION, San Diego, CA (US)

(21) Appl. No.: 12/044,895

(22) Filed: Mar. 7, 2008

Related U.S. Application Data
(60) Provisional application No. 60/905,461, filed on Mar. 7, 2007.
(60) Provisional application No. 60/950,271, filed on Jul. 17, 2007.

(51) Int. Cl. C07H 21/00; A61K 31/7088; A61K 47/26; A61P 37/00

(52) U.S. Cl. 424/278.1; 536/22.1; 536/25.3; 514/44

(57) ABSTRACT

Immunostimulatory agents, including nucleic acids having one or more than one locked nucleic acid (LNA) nucleosides are provided. The nucleic acids may further comprise CpG motifs. The nucleic acids may be double stranded, and may comprise dsRNA.

This application claims the benefit of U.S. Provisional Application Nos. 60/905,461, filed Mar. 7, 2007, and 60/950/271, filed Jul. 17, 2007, both of which are herein incorporated by reference in their entirety.

### FIELD OF INVENTION

The present invention relates to the field of immunology, and immunostimulatory agents. More specifically, the present invention relates to double-stranded locked nucleic acid compositions. The nucleic acids may comprise dsRNA.

### BACKGROUND OF THE INVENTION

The innate immune system has a role as both a ‘first-line’ of defense for an invading pathogen, and also a supporting role for the adaptive immune response. Toll-like receptors (TLRs) are one family of receptors that have a key role in the initiation of both the innate and adaptive immune response. TLRs respond individually to various infectious agent hallmarks, for example, TLR4 is particularly responsive to lipopolysaccharides, TLR9 preferentially responds to methylated nucleic acids, such as nucleic acids comprising a CpG motif, while dsRNAs are the preferred agonist of TLR3.

Double-stranded RNA (dsRNA) is a common replicative intermediate of viral infections. TLR3 initiates a non-specific innate immune response when viral replication occurs in the host, or when a host is exposed to viral replication mimics such as polylC double-stranded RNA. Stimulation of TLR3 leads to activation of NF-kB and subsequent production of inflammatory cytokines including interferons, which in turn enhance the adaptive immune response by stimulating increased expression of MHC class I and class II.

The immunostimulatory characteristic of dsRNAs has been of interest with respect to the development of cancer therapeutics. The use of polylC as an adjuvant and used in combination with therapeutic agents is well known. Furthermore, PolyIC dsRNA has been combined with other agents to improve stability. U.S. Pat. No. 4,346,538 describes polylC complexes comprising relatively high molecular weight polyI:C, poly-L-lysine (a polycationic polypeptide) in a MW range of 13-35 kDa and carboxymethylcellulose (“polylCLC”); and methods of preparation and using such compositions. The use of polylCLC as a therapeutic agent for the treatment of some cancers, some viral diseases such as HIV or Ebola, and also in multiple sclerosis has also been suggested (US Publication 2006/0223742).

Other dsRNAs have also been demonstrated to have some potential as cancer therapeutic agents. For example, dsRNAs in combination with lymphokines have been described as having a synergistic effect as therapeutic agents for treatment of melanoma (EP 0281380). TLR3 agonists, including polylC and polyAU, for use in improved methods in treating cancers have also been described (US 2006/0110746).

Zhu et al (J. Translational Medicine 2007 5:10 doi:10.1186/1479-5876-5-10) describes a combination of polylCLC (administered intramuscularly) and specific tumor immunogens (administered subcutaneously in combination with IFA) as an effective treatment for mice bearing CNS gliomas.

Some PolyI:C compositions have been used in treatment of chronic fatigue syndrome, and in combination with antiviral agents in treatment of HIV infection variants (Thompson et al., Eur J Clin Microbiol Infect Dis. 1996 July; 15(7):580-7; Gillespie et al., In Vivo. 1994 May-June; 8(3):375-81; Strayer et al., Clin Infect Dis 1994 January; 18 Suppl 1:S88-95).

Other specific oligonucleotide motifs have been identified as having immunostimulatory effects, for example CpG dinucleotides. Some unmethylated CpG motifs in DNA are TLR9 agonists, and have been proposed as cancer therapeutics (Krieg A M. 2007 J. Clin Invest 117:1184-94). U.S. Pat. No. 7,148,191 describes an antigenic composition comprising, a polycationic peptide and a nucleic acid comprising inosine and cytosine, for use in combination with a small (6-20 amino acids) antigen. WO 01/93905 describes immunostimulatory oligodeoxynucleotides that exclude CpG motifs, citing side effects such as high systemic TNF-alpha and a lack of specificity.

Therapeutic nucleic acids, including RNAs, may be subject to degradation by the immune response that they stimulate, as part of the innate viral defense response. U.S. Pat. No. 6,194,388 (and references therein) teach that exchanging deoxyribose nucleosides for ribose nucleosides in the nucleic acid compositions is not effective in increasing stability, as the specific form the ribose sugar appears to be required for immune activation. Increasing the dose does not circumvent the stability issues either, as toxicity is dose-dependent.

Adjuvants with improved stability, suitable for co-administration in combination with at least one therapeutic agent, for example, but not limited to, a viral immunogen, and capable of enhancing the immunostimulatory activity of the viral immunogen are desired.

### SUMMARY OF THE INVENTION

The present invention relates to immunostimulatory agents, and provides double-stranded locked nucleic acid (LNA) compositions. The nucleic acids may comprise dsRNA.

It is a further object of the invention to provide an improved dsRNA containing compound.

The present invention also provides a compound of the following formula:

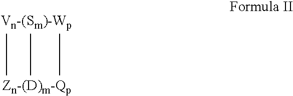

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q is any nucleoside;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside; and

wherein one or more than one of V, S, W, Z, D, and Q, comprises one or more than one locked nucleic acid (LNA) monomer.

The present invention also provides a compound as defined above, wherein B is inosine and D is cytosine.

The present invention pertains to a composition comprising the compound as defined above, a polycationic polypeptide such as polylysine, polyarginine, polyornithine, and carboxymethylcellulose.

The present invention also provides a composition comprising any of the compounds defined above, and an immunogen, for example HspE7.

The present invention also provides a compound of the following formula

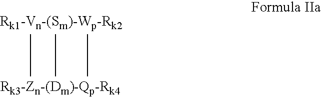

### Where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q may independently be any ribonucleoside connected by an internucleoside linkage group, where V and Z are capable of bonding, and W and Q are capable of bonding.

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand; and

wherein one or more than one of V, S, D, Z, Q, R and W comprises one or more than one LNA monomer.

Formula IIa represents a double-stranded RNA molecule having a 5′, a 3′, or both a 5′ and 3′ overhanging base, and having a first strand Rₖ—Vₙ—(Sₘ)—Wₚ—Rₖ and a second strand Rₖ-Zₙ-(Dₘ)-Qₚ-Rₖ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

The present invention is also directed to a method of treating a subject for a cancer, or a disease or disorder associated with a bacterial or viral pathogen, the method comprising, administering to the subject a compound according to the following formula:

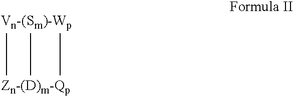

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q is any nucleoside;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytidine, a cytidine-analogue nucleoside, uridine, or a uridine-analogue nucleoside; and

wherein one or more than one of V, W, S, Z, D, and Q, comprises one or more than one locked nucleic acid (LNA) monomer.

The present invention also pertains to the above method, wherein S is inosine and D is cytosine. Furthermore, the compound may be administered along with an immunogen, for example HspE7.

According to another aspect of the invention, there is provided a method of enhancing a subject's immune response to an immunogen, the method comprising administering to a subject a composition comprising an immunogen and a dsRNA comprising an LNA. The immunogen may be a killed whole-organism, a protein, a peptide, a fusion protein, a fusion peptide, a recombinant protein or a recombinant peptide. The immunogen may be HspE7. Examples of dsRNA comprising an LNA include, but are not limited to, Formulae II-VII of the present invention.

The dsRNA comprising molecules of the present invention contain one or more LNAs. These LNA containing dsRNAs exhibit the property of increased stability, while retaining dsRNA activity. LNAs are capable of forming nucleobase specific duplexes and triplexes with single and double stranded nucleic acids. These complexes exhibit higher thermostability than the corresponding complexes formed with normal nucleic acids.

The present invention also provides a compound of the formula

Vₙ—(Sₘ)—Wₚ Formula IVa

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V and W is any nucleoside, ribonucleoside, deoxyribonucleoside, nucleoside analogue, ribonucleoside analogue or deoxyribonucleoside analogue;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside, and;

wherein one or more than one of V, S, and W comprises one or more than one locked nucleic acid (LNA) monomer.

The present invention also provides a compound of the formula

Qₚ-(Dₘ)-Zₙ Formula IVb

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

Z and Q is any nucleoside, ribonucleoside, deoxyribonucleoside, nucleoside analogue, ribonucleoside analogue or deoxyribonucleoside analogue;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

D is cytosine, a cytosine-analogue nucleoside, uracil, or a uracil-analogue nucleoside; and

wherein one or more than one of Z, D, and Q, comprises one or more than one locked nucleic acid (LNA) monomer.

The present invention also provides a method of making a compound of the formula

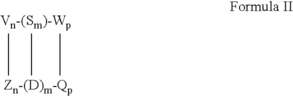

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q is any nucleoside;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside; and

wherein one or more than one of V, S, W, Z, D, and Q, comprises one or more than one LNA monomer, the method comprising:

mixing a molar ratio from about 0.5-1.0 to about 1.0-0.5 of a first oligomer according to the compound of the formula Vₙ—(Sₘ)—Wₚ with a second oligomer according to the compound of the formula Qₚ-(Dₘ)-Zₙ, and annealing said first and second oligomers to form a double-stranded nucleic acid.

The present invention provides a compound of the formula

Rₖ₁—Vₙ—(Sₘ)—Wₚ—Rₖ₂ Formula IVc

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V and W is any nucleoside, ribonucleoside, deoxyribonucleoside, nucleoside analogue, ribonucleoside analogue or deoxyribonucleoside analogue;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

k₁, and k₂, may independently be any integer from 0-10 inclusive, or any integer therebetween;

R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand, wherein one or more than one of V, S, R and W comprises one or more than one locked nucleic acid (LNA) monomer.

The present invention provides a compound of the formula

Rₖ₄-Qₚ-(Dₘ)-Zₙ-Rₖ₃ Formula IVd

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

Z and Q is any nucleoside, ribonucleoside, deoxyribonucleoside, nucleoside analogue, ribonucleoside analogue or deoxyribonucleoside analogue;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

D is cytosine, a cytosine-analogue nucleoside, uracil, or a uracil-analogue nucleoside;

k₃, and k₄ may independently be any integer from 0-10 inclusive, or any integer therebetween;

R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand, wherein one or more than one of R, Z, D, and Q, comprises one or more than one locked nucleic acid (LNA) monomer.

The present invention provides a method of making a compound of the formula

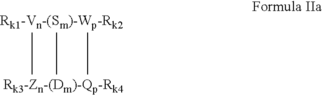

where

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q is any nucleoside;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage group to the geminal nucleoside, or R may be absent, the method comprising mixing a molar ratio from about 0.5-1.0 to about 1.0-0.5 of a first oligomer according to the compound of the formula Rₖ—Vₙ—(Sₘ)—Wₚ—Rₖ with a second oligomer according to the compound of formula Rₖ-Qₚ-(Dₘ)-Zₙ-Rₖ, and annealing said first and second oligomers to form a double-stranded nucleic acid.

The present invention provides a compound of formula:

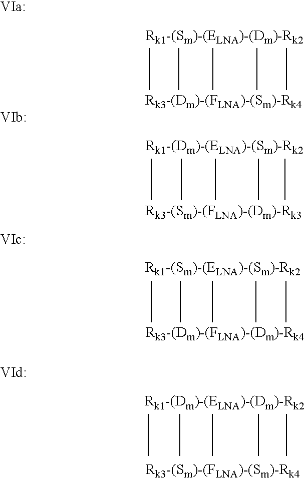

where

E_{LNA} is CpG or a CpG motif, where one or more than one of the nucleosides, C, G, comprising the CpG or the CpG motif is an LNA;

F_{LNA} is CpG or a CpG motif, where one or more than one of the nucleosides, C, G, comprising the CpG or the CpG motif is an LNA;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage.

The present invention also provides a compound of any one of the formula:

Rₖ₃—(Sₘ)-(E_{LNA}) VIIa

Rₖ₂-(Dₘ)-(F_{LNA}) VIIb

Rₖ₃-(Dₘ)-(E_{LNA}) VIIc

Rₖ₁—(Sₘ)—(F_{LNA}) VIId

(E_{LNA})-(Sₘ)—Rₖ₃ VIIe

(F_{LNA})-(Dₘ)-Rₖ₁ VIIf

(E_{LNA})-(Dₘ)-Rₖ₂ VIIg

(F_{LNA})—(Sₘ)—Rₖ₁ VIIh

Where:

E_{LNA} is CpG or a CpG motif, where one or more than one of the nucleosides, C, G, comprising the CpG or the CpG motif is an LNA;

F_{LNA} is CpG or a CpG motif, where one or more than one of the nucleosides, C, G, comprising the CpG or the CpG motif is an LNA;

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage

According to some aspects of the invention,

E_{LNA} is SEQ ID NO: 23, and

F_{LNA} is SEQ ID NO: 24.

The present invention provides a compound of any one of the formula:

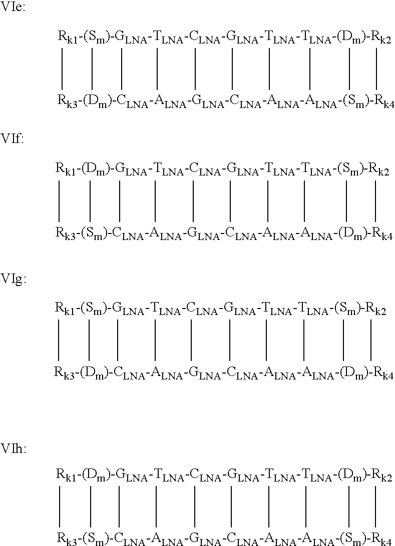

where

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage

The present invention provides a compound of any one of the formula:

Rₖ₁—(Sₘ)-G_{LNA}-T_{LNA}-C_{LNA}-G_{LNA}-T_{LNA}-T_{LNA}-(Dₘ)-Rₖ₂ VIi

Rₖ₃-(Dₘ)-C_{LNA} A_{LNA} G_{LNA} C_{LNA} A_{LNA} A_{LNA}-(Sₘ)—Rₖ₄ VIj

Rₖ₁-(Dₘ)-G_{LNA}-T_{LNA}-C_{LNA}-G_{LNA}-T_{LNA}-T_{LNA}-(Sₘ)—Rₖ₂ VIk

Rₖ₃—(Sₘ)—C_{LNA} A_{LNA} G_{LNA} C_{LNA} A_{LNA} A_{LNA}-(Dₘ)-Rₖ₄ VIl

Rₖ₁—(Sₘ)-G_{LNA}-T_{LNA}-C_{LNA}-G_{LNA}-T_{LNA}-T_{LNA}-(Sₘ)—Rₖ₂ VIm

Rₖ₃-(Dₘ)-C_{LNA} A_{LNA} G_{LNA} C_{LNA} A_{LNA} A_{LNA}-(Dₘ)-Rₖ₄ VIn

Rₖ₁-(Dₘ)-G_{LNA}-T_{LNA}-C_{LNA}-G_{LNA}-T_{LNA}-T_{LNA}-(Dₘ)-Rₖ₂ VIo

Rₖ₃—(Sₘ)—C_{LNA} A_{LNA} G_{LNA} C_{LNA} A_{LNA} A_{LNA}-(Sₘ)—Rₖ₄ VIp

where:

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage

The present invention provides a method (A) of making a compound of the formula

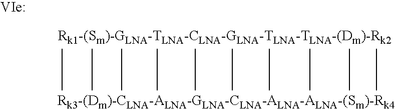

where:

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage

the method comprising:

mixing a molar ratio from about 0.5:1.0 to about 1.0:0.5, of a first oligomer according to the formula:

Rₖ₁—(Sₘ)-G_{LNA}-T_{LNA}-C_{LNA}-G_{LNA}-T_{LNA}-T_{LNA}-(Dₘ)-Rₖ₂ VIi

with a second oligomer according to the formula

Rₖ₃-(Dₘ)-C_{LNA} A_{LNA} G_{LNA} C_{LNA} A_{LNA} A_{LNA}-(Sₘ)—Rₖ₄ VIj

and annealing said first and second oligomers to form a double-stranded nucleic acid.

The present invention provides a method (B) of making a compound of the formula

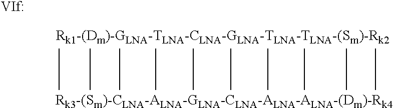

where:

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k may independently be any integer from 0-10 inclusive, or any integer therebetween;

R may independently be any ribonucleoside connected by an internucleoside linkage

the method comprising:

mixing a molar ratio from about 0.5:1.0 to about 1.0:0.5 of a first oligomer according to the compound of the formula:

Rₖ₁-(Dₘ)-G_{LNA}-T_{LNA}-C_{LNA}-G_{LNA}-T_{LNA}-T_{LNA}-(Sₘ)—Rₖ₂ VIk

with a second oligomer according to the compound of the formula

Rₖ₃—(Sₘ)—C_{LNA} A_{LNA} G_{LNA} C_{LNA} A_{LNA} A_{LNA}-(Dₘ)-Rₖ₄ VIl

and annealing said first and second oligomers to form a double-stranded nucleic acid.

The present invention provides a method (C) of making a compound of the formula

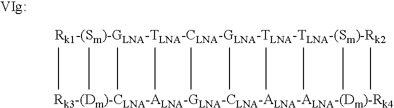

where:

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage

the method comprising:

mixing a molar ratio from about 0.5:1.0 to about 1.0:0.5, of a first oligomer according to the compound of the formula

Rₖ₁—(Sₘ)-G_{LNA}-T_{LNA}-C_{LNA}-G_{LNA}-T_{LNA}-T_{LNA}-(Sₘ)—Rₖ₂ VIm

with a second oligomer according to the compound of the formula

Rₖ₃-(Dₘ)-C_{LNA} A_{LNA} G_{LNA} C_{LNA} A_{LNA} A_{LNA}-(Dₘ)-Rₖ₄ VIn

and annealing said first and second oligomers to form a double-stranded nucleic acid.

The present invention provides a method (D) of making a compound of the formula

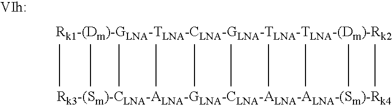

where:

m is any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k may be any integer from 0-10 inclusive, or any integer therebetween;

R may independently be any ribonucleoside connected by an internucleoside linkage

the method comprising:

mixing a molar ratio form about 0.5:1.0 to about 1.0:0.5 of a first oligomer according to the compound of the formula

Rₖ₁-(Dₘ)-G_{LNA}-T_{LNA}-C_{LNA}-G_{LNA}-T_{LNA}-T_{LNA}-(Dₘ)-Rₖ₂ VIo

with a second oligomer according to the compound of the formula

Rₖ₃—(Sₘ)—C_{LNA} A_{LNA} G_{LNA} C_{LNA} A_{LNA} A_{LNA}-(Sₘ)—Rₖ₄ VIp

and annealing said first and second oligomers to form a double-stranded nucleic acid.

The present invention also provides for a compound according to the formula:

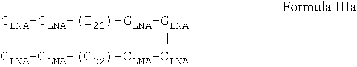

The present invention also provides for a method of making a compound according to Formula IIIa, the method comprising combining of oligomers of each of SEQ ID NO: 1 and SEQ ID NO: 2 and permitting the oligomers to anneal to provide the double-stranded compound of Formula IIIa.

The present invention also provides for compounds according to the formulae:

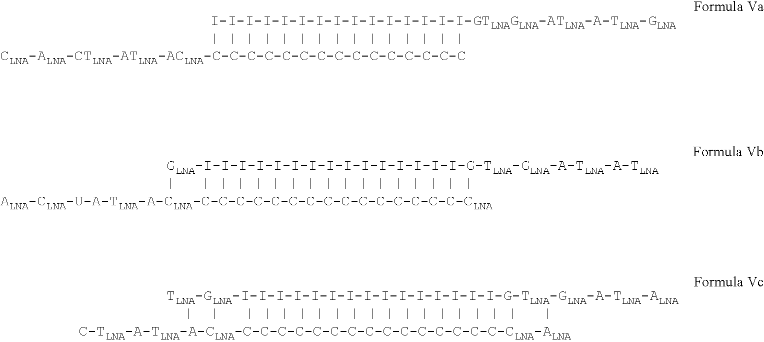

The present invention also provides for methods of making compounds according to Formula Va, Vb and Vc, the method comprising combining oligomers according to SEQ ID NO: 3 and SEQ ID NO: 4, or SEQ ID NO:5 and SEQ ID NO: 6 or SEQ ID NO: 7 and SEQ ID NO: 8; and permitting the oligomers to anneal to produce the double-stranded nucleic acid compounds shown in Formula Va, Vb and Vc, respectively.

(SEQ ID NO: 3)

(I₁₅)-G-T_{LNA}-G_{LNA}-A-T_{LNA}-A-T_{LNA}-G_{LNA}

(SEQ ID NO: 4)

(C₁₅)-C_{LNA}-A-T_{LNA}-A-T_{LNA}-C-A_{LNA}-C_{LNA}

(SEQ ID NO: 5)

G_{LNA}-(I₁₅)-G-T_{LNA}-G_{LNA}-A-T_{LNA}-A-T_{LNA}

(SEQ ID NO: 6)

C_{LNA}-(C₁₅)-C_{LNA}-A-T_{LNA}-A-U-C_{LNA}-A_{LNA}

(SEQ ID NO: 7)

T_{LNA}-G_{LNA}-(I₁₅)-T_{LNA}-T_{LNA}-A-T_{LNA}-A_{LNA}

(SEQ ID NO: 8)

A_{LNA}-C_{LNA}-(C₁₅)-C_{LNA}-A-T_{LNA}-A-T_{LNA}-C_{LNA}

The present invention also provides for double-stranded oligomers with 3′ unpaired ends. The present invention also provides for methods of making double-stranded oligomers with 3′ unpaired ends, the method comprising combining oligomers according to SEQ ID NO: 9 and SEQ ID NO: 10, or SEQ ID NO:11 and SEQ ID NO: 12, or SEQ ID NO: 11 and SEQ ID NO: 25, may be combined and permitted to anneal to produce the double-stranded nucleic acid compounds shown in Formula Vd and Ve, respectively.

| | |
|---|---|
| G_{LNA}-G_{LNA}-(I)₁₅-(A)₁₅ | (SEQ ID NO: 9) |
| (C)₁₅-C_{LNA}-C_{LNA}-(U)₁₅ | (SEQ ID NO: 10) |
| G_{LNA}-G_{LNA}-(I)₁₀-(A)₁₀ | (SEQ ID NO: 11) |
| (U)₁₀-C_{LNA}-C_{LNA}-(C)₁₀ | (SEQ ID NO: 12) |
| (C)₁₀-C_{LNA}-C_{LNA}-(U)₁₀ | (SEQ ID NO: 25) |

The present invention also provides for double-stranded oligomers comprising CpG motifs. The present invention also provides for methods of making double-stranded oligomers comprising combining oligomers according to SEQ ID NO: 13 and SEQ ID NO: 14, or SEQ ID NO: 15 and SEQ ID NO: 16, or SEQ ID NO: 17 and SEQ ID NO: 18, or SEQ ID NO: 19 and SEQ ID NO: 20, or SEQ ID NO: 21 and SEQ ID NO: 22 and permitting the oligomers to anneal, to produce the dsRNA compound according to Formula VIg, VIh, VIi, VIj and VIk.

(SEQ ID NO: 13)

G_{LNA}-G_{LNA}-(I)₁₅-G_{LNA}-T_{LNA}-C_{LNA}-G_{LNA}-T_{LNA}-T_{LNA}-
(I)₁₅-G_{LNA}-G_{LNA}

(SEQ ID NO: 14)

C_{LNA}-C_{LNA}-(C)₁₅-A_{LNA}-A_{LNA}-C_{LNA}-G_{LNA}-A_{LNA}-C_{LNA}-
(C)₁₅-C_{LNA}-C_{LNA}

(SEQ ID NO: 15)

G_{LNA}-G_{LNA}-(I)₁₅-G_{LNA}-T_{LNA}-C_{LNA}-G_{LNA}-T_{LNA}-T_{LNA}-

(SEQ ID NO: 16)

A_{LNA}-A_{LNA}-C_{LNA}-G_{LNA}-A_{LNA}-C_{LNA}-(C)₁₅-C_{LNA}-C_{LNA}

(SEQ ID NO: 17)

(I)₁₅-G_{LNA}-T_{LNA}-C_{LNA}-G_{LNA}-T_{LNA}-T_{LNA}

(SEQ ID NO: 18)

(C)₁₅-A_{LNA}-A_{LNA}-C_{LNA}-G_{LNA}-A_{LNA}-C_{LNA}

(SEQ ID NO: 19)

G_{LNA}-(I)₁₅-G_{LNA}-T_{LNA}-C_{LNA}-G_{LNA}-T_{LNA}-T_{LNA}-

(SEQ ID NO: 20)

C_{LNA}-(C)₁₅-A_{LNA}-A_{LNA}-C_{LNA}-G_{LNA}-A_{LNA}-C_{LNA}

(SEQ ID NO: 21)

C_{LNA}-G_{LNA}-(I)₁₅-G_{LNA}-T_{LNA}-C_{LNA}-G_{LNA}-T_{LNA}-T_{LNA}

(SEQ ID NO: 22)

G_{LNA}-C_{LNA}-(C)₁₅-A_{LNA}-A_{LNA}-C_{LNA}-G_{LNA}-A_{LNA}-C_{LNA}

The compounds of the present invention as described above contain CpG motifs that comprise one or more than one LNA. These LNA containing nucleic acids exhibit the property of increased stability, while retaining CpG-associated activity. LNAs are capable of forming nucleobase specific duplexes and triplexes with single and double stranded nucleic acids. These complexes exhibit higher thermostability than the corresponding complexes formed with normal nucleic acids.

According to some aspects of the invention in the compound defined by any one of Formula VIa to Formula VId, Formula VIIa to Formula VIIh or Formula VIIIa to Formula VIIIh,

E_{LNA} is SEQ ID NO: 23, and

F_{LNA} is SEQ ID NO: 24.

According to some aspects of the invention in the compound defined by any one of Formula II, Formula Ia to Formula IIe, Formula IVa to Formula IVd, Formula VIa to Formula VId, Formula VIe to Formula VIh, Formula VIi to Formula VIp, Formula VIIa to Formula VIIh, Formula VIIIa to Formula VIIIh, S is inosine and D is cytosine in the compound as defined above.

According to some aspects of the invention, the compound as defined above, by any of Formula II, Formula IIa to Formula IIe, Formula III, Formula IIIa to Formula IIId, Formula IVa to Formula IVd, Formula Va to Formula Vc, Formula VIa to Formula VId, Formula VIe to Formula VIh, Formula VIi to Formula VIp, Formula VIIa to Formula VIIh, Formula VIIIa to Formula VIIIh may further comprise a polycationic polypeptide, including polylysine, polyarginine, polyomithine.

The present invention also provides a composition comprising the compound as defined above (Formula II, Formula Ia to Formula IIe, Formula III, Formula IIIa to Formula IIId, Formula IVa to Formula IVd, Formula Va to Formula Vc, Formula VIa to Formula VId, Formula VIe to Formula VIh, Formula VIi to Formula VIp, Formula VIIa to Formula VIIh, Formula VIIIa to Formula VIIIh) and an immunogen, for example HspE7. The present invention also provides methods of treating a subject, comprising administering a pharmaceutically acceptable amount of the composition to the subject.

According to another aspect of the invention, there is provided a method of enhancing a subject's immune response to an immunogen, the method comprising administering to a subject a composition comprising an immunogen and a compound as defined above by any one of Formula II, Formula Ia to Formula IIe, Formula III, Formula IIIa to Formula IIId, Formula IVa to Formula IVd, Formula Va to Formula Vc, Formula VIa to Formula VId, Formula VIe to Formula VIh, Formula VIi to Formula VIp, Formula VIIa to Formula VIIh, Formula VIIIa to Formula VIIIh. The immunogen may be a killed whole-organism, a protein, a peptide, a fusion protein, a fusion peptide, a recombinant protein or a recombinant peptide. The immunogen may be HspE7.

According to another aspect of the invention, there is provided a compound comprising: a first single-stranded nucleotide polymer comprising from one to 500 inosine, cytosine or combination of inosine and cytosine ribonucleotides and from one to ten locked nucleic acid residues; and a second single-stranded nucleotide polymer comprising from one to 500 inosine, cytosine or combination of inosine and cytosine ribonucleotides and from one to ten locked nucleic acid residues; where the first single-stranded nucleotide polymer and the second single-stranded nucleotide polymer are hydrogen bonded to form a double-stranded nucleic acid, the double-stranded nucleic acid comprising a double-stranded polylC region and a double-stranded region comprising locked nucleic acid residues.

The present invention also provides a compound comprising a first single-stranded nucleotide polymer comprising from one to 500 inosine, cytosine or combination of inosine and cytosine ribonucleotides and a nucleic acid sequence according to SEQ ID NO: 23; and a second single-stranded nucleotide polymer comprising from one to 500 inosine, cytosine or a combination of inosine and cytosine ribonucleotides and a nucleic acid sequence according to SEQ ID NO: 24; where the first single-stranded nucleotide polymer and the second single-stranded nucleotide polymer are hydrogen bonded to form a double-stranded nucleic acid, the double-stranded nucleic acid comprising a double-stranded polylC region and a double-stranded region comprising locked nucleic acid residues.

The present invention further provides an adjuvant or adjuvant composition comprising a first single-stranded nucleotide polymer comprising from one to 500 inosine, cytosine or combination of inosine and cytosine ribonucleotides and a nucleic acid sequence according to SEQ ID NO: 23; and a second single-stranded nucleotide polymer comprising from one to 500 inosine, cytosine or a combination of inosine and cytosine ribonucleotides and a nucleic acid sequence according to SEQ ID NO: 24; where the first single-stranded nucleotide polymer and the second single-stranded nucleotide polymer are hydrogen bonded to form a double-stranded nucleic acid, the double-stranded nucleic acid comprising a double-stranded polylC region and a double-stranded region comprising locked nucleic acid residues.

The present invention further provides an adjuvant or adjuvant composition comprising a first single-stranded nucleotide polymer comprising from one to 500 inosine, cytosine or combination of inosine and cytosine ribonucleotides and from one to ten locked nucleic acid residues; and a second single-stranded nucleotide polymer comprising from one to 500 inosine, cytosine or combination of inosine and cytosine ribonucleotides and from one to ten locked nucleic acid residues; where the first single-stranded nucleotide polymer and the second single-stranded nucleotide polymer are hydrogen bonded to form a double-stranded nucleic acid, the double-stranded nucleic acid comprising a double-stranded polylC region and a double-stranded region comprising locked nucleic acid residues.

The present invention further provides an adjuvant or adjuvant composition having dual-receptor agonist activity for TLR3 and TLR9 receptors, the adjuvant or adjuvant composition comprising a first single-stranded nucleotide polymer comprising from one to 500 inosine, cytosine or combination of inosine and cytosine ribonucleotides and a nucleic acid sequence according to SEQ ID NO: 23; and a second single-stranded nucleotide polymer comprising from one to 500 inosine, cytosine or a combination of inosine and cytosine ribonucleotides and a nucleic acid sequence according to SEQ ID NO: 24; where the first single-stranded nucleotide polymer and the second single-stranded nucleotide polymer are hydrogen bonded to form a double-stranded nucleic acid, the double-stranded nucleic acid comprising a double-stranded polylC region and a double-stranded region comprising locked nucleic acid residues.

This summary of the invention does not necessarily describe all features of the invention. Other aspects and features of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows double-stranded nucleic acid compounds according to Formula Vd and Ve, in accordance with an embodiment of the present invention.

FIG. 2 shows a double-stranded nucleic acid according to Formula VIg to Formula VIk, in accordance with an embodiment of the present invention.

FIG. 3 shows a double-stranded nucleic acid comprising a polyA and polyU region, in accordance with an embodiment of the present invention.

### DETAILED DESCRIPTION

The present invention relates to immunostimulatory agents, and provides double-stranded locked nucleic acid (LNA) compositions. The nucleic acids may comprise dsRNA.

Use of examples in the specification, including examples of terms, is for illustrative purposes only and is not intended to limit the scope and meaning of the embodiments of the invention herein.

The present invention provides a composition comprising polyl and polyc, or polyA and polyU oligonucleotide polymers, wherein each of the oligonucleotide polymer comprises at least one locked nucleic acid (LNA) residue. The dsRNA may be comprised of about equimolar quantities of polyI and polyC oligonucleotide polymers (polyI:C), or about equimolar quantities of polyA and polyU oligonucleotide polymers (polyA:U).

The present invention further provides a composition comprising a pair of oligonucleotide polymers, each comprising a mixture of I (inosine) and C (cytosine) nucleosides, wherein the I and C nucleosides in the pair of oligonucleotide polymers are arranged so as to permit the pair of oligonucleotide polymers to hybridize to form a double-stranded molecule.

The present invention further provides a composition comprising polyI and polyC, or polyA and polyU oligonucleotide polymers, wherein each of the oligonucleotide polymer comprises at least one CpG motif and at least one locked nucleic acid (LNA) residue. The CpG motif may comprise at least one LNA residue. The dsRNA may be comprised of about equimolar quantities of polyl and polyC oligonucleotide polymers (polyI:C), about or equimolar quantities of polyA and polyU oligonucleotide polymers (polyA:U).

The present invention further provides a composition comprising oligonucleotide polymers comprising at least one CpG motif and at least one LNA residue, and a combination of I and C residues, or combination A and U residues. The oligonucleotide polymers may hybridize and form double-stranded molecules, for example double-stranded RNA (dsRNA). For example the dsRNA that comprise a CpG motif and having one or more than one LNA may be a polyI:C compound comprising one or more than one LNA. The dsRNA may be comprised of about equimolar quantities of polyI and polyC oligonucleotide polymers (polyI:C), or about equimolar quantities of polyA and polyU oligonucleotide polymers (polyA:U). In another example, the oligonucleotide polymers may comprise a CpG motif comprising one, or more than one LNA, and a mixture of I and C nucleosides, or a mixture of A and U nucleosides, wherein the CpG motif and the I and C nucleosides of each oligonucleotide in the pair are arranged so as to hybridize to form a double-stranded molecule.

The dsRNA of the present invention, that comprise at least one CpG motif and one or more than one LNA, may be used for a variety of purposes, for example, but not limited to their use as adjuvants, or as immunostimulatory agents, or as therapeutic agents. For example the dsRNA that comprise at least one CpG motif and one or more than one LNA may be a polyI:C compound comprising one or more than one LNA.

Immunostimulatory agents are compounds or compositions that initiate an immune response, or provide a catalytic effect in initiating an immune response. The immune response may be solely an innate (or non-adaptive) immune response, such as inducing the production and secretion of cytokines (for example interferons, interleukins, colony stimulating factors and the like) which in turn incite phagocytic cells to migrate and ingest foreign immunogens nonspecifically and present the immunogens for recognition by the adaptive immune system. Alternatively, the immune response may be an adaptive immune response, in response to the presence of particular immunogens (such as those presented by an phagocytic cell, also referred to as an antigen-presenting cell).

Use of the term ‘a’ or ‘an’ includes both singular and plural references.

An adjuvant is an immunostimulatory agent that has no specific immunogenic effect by itself, but stimulates the immune system to increase or enhance the response to a specific immunogen, or group of immunogens. The ability of an immunogen to induce a response of the innate or adaptive immune system is referred to as the “biological activity” of the immunogen. An adjuvant may mediate, augment or stimulate the biological activity of an immunogen. In some examples, the immunogen may have very little or negligible biological activity in the absence of an adjuvant.

The biological activity of an immunogen may be measured by any of several assays known in the art. For example, induction of antigen-specific CD8-positive T lymphocytes may be quantified through use of an ELISPOT assay (Asai et al 2000 Clin. Diag. Lab Immunol 7:145-154). Other versions of an ELISPOT assay may be used for other cytokines, see, for example, Kalyzhny et al 2005. Methods Mol Biol 302:15-31; Ott, et al. J. Immunol. Methods. 2004 Feb. 15; 285(2):223-35; Forsthuber, et al. Science, 271: 1728-1730. Other T-cell assays that may be useful for monitoring a response to an immunogen include intracellular cytokine flow cytometry, proliferation assays, antibody microarrays, and the like. See, for example Nagorsen et al 2004. Expert Opin Biol Ther 4:1677-84, or Handbook of Experimental Immunology, Vols. I-IV, D. M. Weir and C. C. Blackwell, eds., 1986, Blackwell Scientific Publications. Interferon-α and β may be quantified with an Interferon ELISA kit (Kim et al 2004. Nature Biotechnology 22:321-325). Multiplexed assays, for example, bead-based systems (Luminex, Panomics and the like) allow for simultaneous quantification of a plurality of cytokines. Examples of cytokines include IL-1α, IL-1β, IL-2, II-4, IL-5, IL-6, IL-7, IL-8, IL-9, IL-10, IL-12 (p70), IL-13, IL-15, IL-17, IL-18, IFNα, IFNβ, IFNγ, GM-CSF, TNFα, G-CSF, MIP-1α, MIP-1β, MCP-1, EOTAXIN, RANTES, FGF-basic, VEGF and the like. For clarity, the term ‘cytokine’ includes alternative nomenclatures such as lymphokines, interleukins, or chemokines

The terms “subject” and “patient” may be used interchangeably. A “subject” refers to an animal, or a mammal, including, but not limited to, a mouse, rat, dog, cat, pig, or primate, including but not limited to a monkey, chimpanzee or human. The subject may be immunologically naïve with respect to a particular immunogen or group of immunogens, or the subject may have been previously exposed to a particular immunogen or group of immunogens. Previous exposure may have resulted from, for example, deliberate immunization with a particular immunogen or group of immunogens, exposure to an infectious agent comprising a particular immunogen or group of immunogens, or cross-reactive exposure to a first immunogen or group of immunogens, that allows an immune response to a second immunogen or group of immunogens. The second immunogen or group of immunogens may be similar to, the same as, or different from the first immunogen or group of immunogens.

As used herein, the term “LNA-modified oligonucleotide” includes to any oligonucleotide either fully or partially modified with one or more LNA monomer. Thus, an LNA-modified oligonucleotide may be composed entirely by LNA monomers, or a LNA-modified oligonucleotide may comprise one LNA monomer.

The term “DNA monomer” refers to a deoxyribose sugar bonded to a nitrogenous base, while the term “RNA monomer” refers to a ribose sugar bonded to a nitrogenous base. Examples of DNA monomers that may comprise compositions according to various embodiments of the present invention include, but are not limited to, deoxyadenosine, deoxyguanosine, deoxythymidine, deoxyuridine, deoxycytidine, deoxyinosine and the like. Examples of RNA monomers that may comprise compositions according to various embodiments of the present invention include, but are not limited to, adenosine, guanosine, 5-methyluridine, uridine, cytidine, inosine, and the like. Other DNA or RNA monomers according to various embodiments of the present invention may comprise other nitrogenous bases, as are known in the art.

As used herein, the term “LNA monomer” typically refers to a nucleoside having a 2′-4′ cyclic linkage as described in U.S. Pat. No. 6,268,490, U.S. Pat. No. 6,794,499, U.S. Pat. No. 7,034,133 (each of which are incorporated herein by reference). Bicyclic nucleosides (see below) may provide conformational restriction to the oligonucleotide, and may provide varying hybridization or stability profiles compared to unmodified oligonucleotides.

The term ‘nucleoside’ refers to a molecule of ribose or deoxyribose sugar bonded through carbon-1 of the sugar ring to a nitrogenous base. Examples of nitrogenous bases include purines such as adenine, guanine, 6-thioguanine, hypoxanthine, xanthine, and pyrimidines such as cytosine, thymine and uracil. Examples of purine nucleosides include adenosine (A), guanosine (G), inosine (I), 2′-O-methyl-inosine, 2′-O-methyl-adenosine, 2′-O-methyl-guanine, 2-chlorodeoxyadenosine, 7-halo-7-deaza-adenosine, 7-halo-7-deaza-guanine, 7-propyne-7-deaza adenosine, 7-propyne-7-deaza-guanine, 2-amino-adenosine, 7-deazainosine, 7-thia-7,9-dideazainosine, formycin B, 8-Azainosine, 9-deazainosine, allopurinol riboside, 8-bromo-inosine, 8-chloroinosine, 7-deaza-2-deoxy-xanthosine, 7-deaza-8-aza-adenosine, 7-deaza-8-aza-guanosine, 7-deaza-8-aza-deoxyadenosine, 7-deaza-8-aza-deoxyguanosine, 7-deaza-adenosine, 7-deaza-guanosine, 7-deaza-deoxyadenosine, 7-deaza-deoxyguanosine, 8-amino-adenosine, 8-amino-deoxyadenosine, 8-amino-guanosine, 8-amino-deoxyguanosine, 3-deaza-deoxyadenosine, 3-deaza-adenosine, 6-thio-deoxyguanosine, N6-isopentenyladenosine, 1-methyladenosine, 1-methylpseudouridine, 1-methylguanosine, 1-methylinosine, 2,2-dimethylguanosine, 2-methyladenonsine, 2-methylguanosine, N6-methyladenosine, 7-methylguanosine, 2-methylthioN6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, N-((9-beta-D-ribofuranosylpurine-6-yl)carbamoyl)threonine, wybutosine, wybutoxosine and the like, and other purine nucleosides as described in Freier et al 1997 (Nucleic Acids Res. 25:4429-4443), incorporated herein by reference.

Examples of pyrimidine nucleosides include deoxyuridine (dU), uridine (U), cytidine (C), deoxycytidine (dC), thymidine (T), deoxythymidine (dT), 5-fluoro-uracil, 5-bromouracil, 2′-O-methyl-uridine, 2′-O-methyl cytidine, 5-iodouracil, 5-methoxy-ethoxy-methyl-uracil, 5-propynyl deoxyuridine, pseudoisocytidine, 5-azacytidine, 5-(1-propynyl)cytidine, 2′-deoxypseudouridine, 4-thio-deoxythymidine, 4-thio-deoxyuridine, 4-acetylcytidine, 5-(carboxyhydroxymethyl)uridine) 2′-O-methylcytidine, 5-carboxymethylaminomethyluridine, dihydrouridine, 2′-O-methylpseudouridine, 3-methylcytidine, 5-methylcytidine, 5-methylaminomethyluridine, 5-methoxyaminomethyl-2-thiouridine, 5-methoxycarbonylmethyl-2-thiouridine, 5-methoxycarbonylmethyluridine, 5-methoxyuridine, uridine-5-oxyacetic acid-methylester, uridine-5-oxyacedic acid, pseudouridine, 2-thiocytidine, 5-methyl-2-thiouridine, 2-thiouridine, 4-thiouridine, 5-methyluridine, 2′-O-methyl-5-methyluridine, 2′-O-methyluridine, 3-(3-amino-3-carboxy-propyl)uridine and the like, and other substituted pyrimidines as disclosed in Freier, et α1, 1997 (Nucleic Acids Res. 25:4429-4443).

Purine or pyrimidine nucleosides also include phosphoramidite derivatives used in oligonucleotide synthesis using standard methods.

The term nucleoside further includes bicyclic nucleoside analogues according to Formula (I), as described in, for example, U.S. Pat. No. 6,268,490 (which is incorporated by reference):

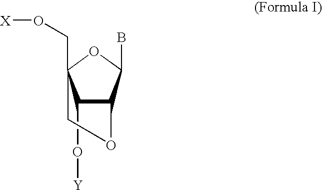

B may be any nitrogenous base, for example a pyrimidine or purine nucleic acid base, or an analogue thereof.

X and Y may be identical or different, and may be any internucleoside linkage group.

Such bicyclic nucleoside analogues may alternately be referred to as “locked nucleic acid monomer” or “locked nucleoside monomer” or “LNA monomer” or “LNA residue”. Methods of synthesis and polymerization of nucleic acid polymers comprising LNA monomers are described in, for example, WO 99/14226, WO 00/56746, WO 00/56748, WO 01/25248, WO 0148190, WO 02/28875, WO 03/006475, WO 03/09547, WO 2004/083430, U.S. Pat. No. 6,268,490, U.S. Pat. No. 6,794,499, U.S. Pat. No. 7,034,133 (each of which are herein incorporated by reference).

Other examples of nucleoside analogues, as disclosed in WO 01/048190 (which is incorporated herein by reference) include non-LNA bicyclic nucleosides, for example, but not limited to:

bicyclo[3.3.0]nucleosides with an additional C-3′,C-5′-ethanobridge;

bicarbocyclo[3.1.0]nucleosides with an additional C-1′,C-6′- or C-6′,C-4′methano bridge

bicyclo[3.3.0]- and [4.3.0]nucleosides containing an additional C-2′,C-3′dioxalane ring synthesised as a dimer with an unmodified nucleoside where the additional ring is part of the internucleoside linkage replacing a natural phosphordiester linkage; dimers containing a bicyclo[3.1.0]nucleoside with a C-2′,C-3′-methano bridge as part of amide- and sulfonamide-type internucleoside linkages;

bicyclo[3.3.0]glucose derived nucleoside analogue incorporated in the middle of a trimer through formacetal internucleoside linkages;

tricyclo-DNA in which two five membered rings and one three membered ring constitute the backbone;

1,5-Anhydrohexitol nucleic acids; and

bicyclic[4.3.0]- and [3.3.0]nucleosides with additional C-2′,C-3′-connected six and five-membered ring.

“Nucleoside” also includes nucleosides having substituted ribose sugars (bicyclic or otherwise). Examples of substituted ribose sugars are described in, for example, Freier, 1997 (Nucleic Acids Res. 25:4429-4443), which is incorporated by reference).

A ‘nucleotide’ refers to a nucleoside having an internucleoside linkage group bonded through the carbon-5 of the sugar ring. An oligonucleotide ‘backbone’ refers to, for example, in a naturally occurring nucleic acid, the alternating ribose/phosphate chain covalently bonded through the carbon-5 and carbon-3 of consecutive sugars, formed by polymerization of a population of nucleotides. This may involve synthetic chemical methods, as are known in the art. See, for example, Gait, pp. 1-22; Atkinson et al., pp. 35-81; Sproat et al., pp. 83-115; and Wu et al., pp. 135-151, in Oligonucleotide Synthesis: A Practical Approach, M. J. Gait, ed., 1984, IRL Press, Oxford; or Molecular Cloning: a Laboratory Manual 3^{rd} edition. Sambrook and Russell. CSHL Press, Cold Spring Harbour, New York (all of which are herein incorporated by reference).

The polymerization may also be enzymatic. LNA nucleoside triphosphates may also be used as substrates for enzymatic polymerization of nucleic acid compounds or compositions according to some embodiments of the invention. LNA nucleosides may be incorporated into an extending nucleic acid polymer by a polymerase, for example a DNA or RNA polymerase, in a PCR reaction or primer extension assay. Examples of suitable polymerases include, but are not limited to, Phusion™ High Fidelity DNA polymerase (Finnzymes), or 9°N_{m™ DNA polymerase. Methods of enzymatic incorporation of LNA nucleosides are described in, for example Veedu R N et al} 2007. Nucleic Acids Symposium 51:29-30 and Veedu R N et al. 2007. ChemBioChem 8:490-492 and Veedu et al 2007. Nucleosides, Nucleotides and Nucleic Acids 26:1207-1210; each of which are incorporated herein by reference.

An internucleoside linkage group refers to a group capable of coupling two nucleosides, as part of an oligonucleotide backbone. Examples of internucleoside linkage groups are described by Praseuth et al (Biochimica et Biophysica Acta 1489:181-206, incorporated herein by reference), including phosphodiester (PO₄—), phosphorothioate (PO3ₛ-), phosphoramidate (N3′-P5′) (PO₃NH) and methylphosphonate (PO₃CH₃), peptidic linkages (“PNA”), and the like.

The terms “nucleotide polymer”, “oligonucleotide”, “oligonucleotide polymer”, “oligonucleotide”, “nucleic acid”, “oligomer” or “nucleic acid polymer” are used interchangeably, and refer to polymers comprising at least two nucleotides. The nucleotide polymer may comprise a single species of DNA monomer, RNA monomer, or may comprise two or more species of DNA monomer, RNA monomers in any combination. Nucleic acid may be single or double-stranded, for example, a double-stranded nucleic acid molecule may comprise two single-stranded nucleic acids that hybridize through base pairing of complementary bases.

A “polyl” oligonucleotide includes a majority of inosine, inosine-analogue nucleosides, or a combination thereof. Inosine-analogue nucleosides include, for example, 7-Deazainosine, 2′-O-methyl-inosine, 7-thia-7,9-dideazainosine, formycin B, 8-Azainosine, 9-deazainosine, allopurinol riboside, 8-bromo-inosine, 8-chloroinosine and the like.

A “polyC” oligonucleotide includes a majority of cytidine, cytidine-analogue nucleosides, or a combination thereof. Cytidine-analogue nucleosides include, for example, 5-methylcytidine, 2′-O-methyl-cytidine, 5-(1-propynyl)cytidine, and the like.

A “polyA” oligonucleotide includes a majority of adenosine, adenosine-analogue nucleosides, or a combination thereof. Adenosine-analogue nucleosides include, for example, 2-amino-ademosine, 2′-O-methyl-adenosine, 2-amino-deoxyademosine, 7-deaza-2′-adenosine, 7-deaza-2′-deoxyadenosine, and the like.

A “polyU” oligonucleotide includes a majority of uridine, uridine-analogue nucleosides, or a combination thereof. Uridine-analogue nucleosides include, for example deoxyuridine (dU), cytidine (C), deoxycytidine (dC), thymidine (T), deoxythymidine (dT), 5-fluoro-uracil, 5-bromouracil, 2′-O-methyl-uridine, 5-iodouracil, 5-methoxy-ethoxy-methyl-uracil, 5-propynyl deoxyuridine, and the like.

A “CpG motif” or a “CpG element” or a “CpG site” refers to a nucleotide motif comprising a cytosine nucleoside occurring adjacent to a guanine nucleoside in a nucleic acid. The nucleosides C and G are separated by a phosphate which links the two together in a conventional 5′-3′ nucleosidic linkage. A CpG motif may be described generally as XnCpGXn, where X is any nucleoside and n is any number from 1 to about 500 or any amount therebetween, for example from about 1 to about 300 or any amount therebetween, from amount 1 to about 250 or any amount therebetween, from about 1 to about 200 or any amount therebetween, from about 1 to about 150 or any amount therebetween, or from 1, 5, 10, 20, 30, 40, 50, 60, 70, 80, 90, 100, 125, 150, 175, 200, 225, 250, 275, 300, 325, 250, 275, 400, 425, 250, 475, 500 or any amount therebetween. As described herein, it is preferred that one or more than one of the nucleosides, C, G, within the CpG motif is an LNA.

The strands of double-stranded nucleic acid molecules, including dsRNA, interact in an ordered manner through hydrogen bonding—also referred to as ‘Watson-Crick’ base pairing. Variant base-pairing may also occur through non-canonical hydrogen bonding includes Hoogsteen base pairing. Under some thermodynamic, ionic or pH conditions, triple helices may occur, particularly with ribonucleic acids. These and other variant hydrogen bonding or base-pairing are known in the art, and may be found in, for example, Lehninger—Principles of Biochemistry, 3^{rd} edition (Nelson and Cox, eds. Worth Publishers, New York), herein incorporated by reference.

PolyI and polyC, or polyA and polyU oligonucleotides according to various embodiments of the invention and under suitable temperature, ionic and pH conditions may form double-stranded complexes through Watson-Crick hydrogen bonding. The particular temperature, ionic and pH conditions suitable for such complex formation are discernable by one of skill in the art—examples of methods, calculations, techniques and the like for discerning such conditions may be found in, for example, Freier, (1997, Nucleic Acids Res. 25:4429-4443; which is incorporated herein by reference). The formation of such double-stranded complexes may alternately be referred to as ‘hybridization’.

Double stranded RNA (dsRNA) molecules according to various embodiments of the invention that contain at least one LNA, are generally described by Formula II:

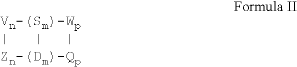

Formula II represents a double-stranded RNA molecule having a first strand Vₙ—(Sₘ)—Wₚ and a second strand Zₙ-(Dₘ)-Qₚ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q is any nucleoside, ribonucleoside, deoxyribonucleoside, nucleoside analogue, ribonucleoside analogue or deoxyribonucleoside analogue;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside; and

wherein one or more than one of V, S, W, Z, D, and Q, comprises one or more than one locked nucleic acid (LNA) monomer.

Double stranded RNA (dsRNA) molecules according to various embodiments of the invention that contain at least one LNA and further comprising R, are generally described by Formula IIa:

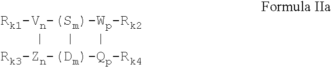

Formula IIa represents a double-stranded RNA molecule having a 5′, a 3′, or both a 5′ and 3′ overhanging base, and having a first strand Rₖ₁—Vₙ—(Sₘ)—Wₚ—Rₖ₂ and a second strand Rₖ₃-Zₙ-(Dₘ)-Qₚ-Rₖ₄, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q may independently be any ribonucleoside connected by an internucleoside linkage group, where V and Z are capable of bonding, and W and Q are capable of bonding.

m may be any integer from 1 to 500, or from 10 to 50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand; and

wherein one or more than one of R, V, S, W, Z, D, and Q, comprises one or more than one LNA monomer.

Nucleic Acids Comprising polyI:C

The presenting invention also provides a dsRNA compound of Formula II where S and D are I and C as defined below (Formula IIb):

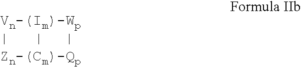

Formula IIb represents a double-stranded RNA molecule having a first strand Vₙ—(Iₘ)—Wₚ and a second strand Zₙ-(Cₘ)-Qₚ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q may independently be any nucleoside connected by an internucleoside linkage group, where V and Z are capable of bonding, and W and Q are capable of bonding;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

I is inosine, or any inosine-analogue nucleoside connected to V, W and to geminal inosine or inosine-analogues nucleoside by an internucleoside linkage group;

C is cytosine, or any cytosine-analogue nucleoside connected to V, W and to geminal cytosine, or any cytosine-analogues nucleoside by an internucleoside linkage group; and

wherein one or more than one of V, I, W, Z, C, and Q, comprises one or more than one LNA monomer.

Alternate dsRNA molecules of the present invention, include a compound of Formula II, where S and D are I and C, and further comprising R, as defined below (Formula IIc):

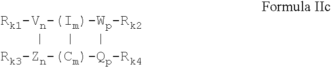

Formula IIc represents a double-stranded RNA molecule having a 5′, a 3′, or both a 5′ and 3′ overhanging base, and having a first strand Rₖ—Vₙ—(Iₘ)—Wₚ—Rₖ and a second strand Rₖ-Zₙ-(Cₘ)-Qₚ-Rₖ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

n is any integer from 0 to 10, or any amount therebetween, with the proviso that if n=0, p 1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q may independently be any ribonucleoside connected by an internucleoside linkage group, where V and Z are capable of bonding, and W and Q are capable of bonding.

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

I may be inosine, or any inosine-analogue nucleoside connected to V, W and to geminal inosine or inosine-analogues by an internucleoside linkage group.

C may be cytosine, or any cytosine-analogue ribonucleoside connected to V, W and to geminal cytosine, or any cytosine-analogues by an internucleoside linkage group bond.

k₁, k₂, k₃, and k₄ may independently be any integer from 0-10 inclusive, or any integer therebetween;

R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand; and

wherein one or more than one of R, V, I, W, Z, C, and Q, comprises one or more than one LNA monomer.

Double stranded RNA (dsRNA) molecules that contain at least one LNA, include a compound of Formula II, where S and D are A and U, as defined below (Formula IId): are generally are also described by Formula IId:

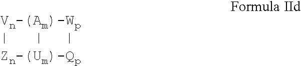

Formula IId represents a double-stranded RNA molecule having a first strand Vₙ-(Aₘ)—Wₚ and a second strand Zₙ-(Uₘ)-Qₚ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q may independently be any nucleoside connected by an internucleoside linkage group, where V and Z are capable of bonding, and W and Q are capable bonding;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

A may be adenosine, or any adenosine-analogue nucleoside connected to V, W and to geminal adenosine or adenosine-analogues by an internucleoside linkage group;

U may be uridine, or any uridine-analogue nucleoside connected to V, W and to geminal uridine, or any uridine-analogues by an internucleoside linkage group; and

wherein one or more than one of R, V, A, W, Z, U, and Q, comprises one or more than one LNA monomer.

Alternate dsRNA molecules of the present invention, include a compound of Formula II, where S and D are A and U, and further comprising R, as defined below (Formula IIe): where at least one nucleoside for the dsRNA is an LNA

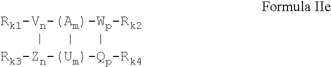

Formula IIe represents a double-stranded RNA molecule having a 5′, a 3′, or both a 5′ and 3′ overhanging base, and having a first strand Rₖ₁—Vₙ—(Iₘ)—Wₚ—Rₖ₂ and a second strand Rₖ₄-Zₙ-(Cₘ)-Qₚ-Rₖ₃, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V, W, Z and Q may independently be any nucleoside connected by an internucleoside linkage group, where V and Z are capable of bonding, and W and Q are capable of bonding.

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

A may be adenosine, or any adenosine-analogue nucleoside connected to V, W and to geminal adenosine or adenosine-analogues by an internucleoside linkage group;

U may be uridine, or any uridine-analogue nucleoside connected to V, W and to geminal uridine, or any uridine-analogues by an internucleoside linkage group; and

k₁, k₂, k₃, and k₄ may independently be any integer from 0-10 inclusive, or any integer therebetween;

R may independently be any nucleoside connected by an internucleoside linkage group to the geminal nucleoside, or R may be absent. In some embodiments, for example, a 5′ R nucleoside of the first strand is capable of bonding with a 3′ R nucleoside of the second strand; and

wherein one or more than one of R, V, A, W, Z, U, and Q, comprises one or more than one LNA monomer.

Compounds according to Formula II, Ia, IIb, IIc, IId, IIe may comprise one or more than one LNA molecule at one or more than one of the R, V, W, Z, Q. For example one or more than one LNA molecule may be positioned at the 5′ end of Formula II, Ia, IIb, IIc, IId or IIe, within V, Q, or both V and Q, one or more than one LNA molecule may be positioned at the 3′ end of Formula II, Ia, IIb, IIc, IId or IIe within Z, W, or both Z and W, or one or more than one LNA molecule may be positioned at the 5′ and the 3′ ends of Formula II, IIa, IIb, IIc, IId or IIe within V, W, Z, Q or a combination thereof.

The present invention also provides a compound according to Formula II, Ia, IIb, IIc, IId or IIe where V and W are LNA nucleosides (V_{LNA}, W_{LNA}, respectively), Z and Q are LNA nucleosides (Z_{LNA}, Q_{LNA}, respectively), I is inosine, C is cytidine, n and p is 2, m is as defined above, and may be from about 1 to about 500 or any amount therebetween, for example m is from about 10 to about 50 or any amount therebetween, for example m is about 1, 2, 5, 7, 10, 12, 14, 16, 17, 18, 19, 20, 21, 22, 23, 24, 25, 26, 27, 28, 29, 30, 32, 35, 40, 45, 50, 60, 70, 80 90, 100 or any amount therebetween, for example m may be 18, 19, 20, 21, 22, 23, 24, 25, and the internucleoside linkage groups therebetween are phosphodiester. A non-limiting example of this compound is shown in Formula III:

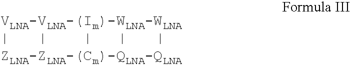

A non-limiting example of a dsRNA of the present invention may be as shown in any one of Formula IIIa, IIIb, IIIc, or IIId, where G is a guanosine nucleoside, C is a cytidine nucleoside and m is 22:

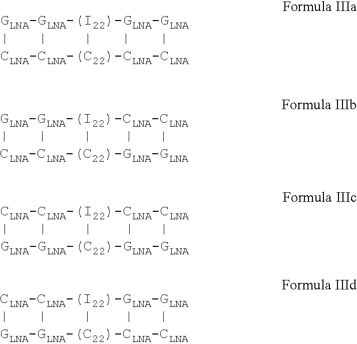

Single stranded nucleic acid molecules, or single-stranded RNA (ssRNA) molecules according to various embodiments of the invention that comprise at least one LNA, are generally described by Formula IVa:

Vₙ—(Sₘ)—Wₚ Formula IVa

Formula IVa represents a single-stranded nucleic acid molecule having a configuration Vₙ—(Sₘ)—Wₚ, represented in a 5′ to 3′ direction (left to right) where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V and W is any nucleoside, ribonucleoside, deoxyribonucleoside, nucleoside analogue, ribonucleoside analogue or deoxyribonucleoside analogue;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside, and;

wherein one or more than one of V, S, and W comprises one or more than one locked nucleic acid (LNA) monomer.

Single stranded nucleic acid molecules, or single-stranded RNA (ssRNA) molecules according to various embodiments of the invention that comprise at least one LNA, are generally described by Formula IVb:

Qₚ-(Dₘ)-Zₙ Formula IVb

Formula IVb represents a single-stranded RNA molecule having a first strand Qₚ-(Dₘ)-Zₙ, represented in a 5′ to 3′ direction (left to right)

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

Z and Q is any nucleoside, ribonucleoside, deoxyribonucleoside, nucleoside analogue, ribonucleoside analogue or deoxyribonucleoside analogue;

m is any integer from 1 to 500, or any amount therebetween;

D is cytosine, a cytosine-analogue nucleoside, uracil, or a uracil-analogue nucleoside; and

wherein one or more than one of Z, D, and Q, comprises one or more than one locked nucleic acid (LNA) monomer.

In some embodiments of the invention, compositions may comprise single-stranded RNA molecules according to Formula IVa or Formula IVb, or both Formula IVa and Formula IVb in various molar ratios. For example, in some embodiments, single stranded RNA molecules according to Formula IVa and Formula IVb may be combined in about equimolar ratios. Some, none or all single-stranded RNA molecules according to Formula IVa and Formula IVb may hybridize with another complementary single-stranded RNA molecule to form double-stranded RNA molecules.

In other embodiments, single stranded RNA molecules according to Formula IVa may be combined in a composition with single stranded RNA molecules according to Formula IVb in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVa or Formula IVb may hybridize with another complementary single-stranded RNA molecule to form double-stranded RNA molecules.

In other embodiments, single stranded RNA molecules according to Formula IVb may be combined in a composition with single stranded RNA molecules according to Formula IVa in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVa or Formula IVb may hybridize with another complementary single-stranded RNA molecule to form double-stranded RNA molecules.

Single stranded nucleic acid molecules, or single-stranded RNA (ssRNA) molecules according to various embodiments of the invention that comprise at least one LNA, are generally described by Formula IVc:

Rₖ₁—Vₙ—(Sₘ)—Wₚ—Rₖ₂ Formula IVc

Formula IVc represents a single-stranded nucleic acid molecule having a configuration Rₖ₁—Vₙ—(Sₘ)—Wₚ—Rₖ₂, represented in a 5′-3′ direction (left to right)

where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

V and W is any nucleoside, ribonucleoside, deoxyribonucleoside, nucleoside analogue, ribonucleoside analogue or deoxyribonucleoside analogue;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

k₁, and k₂ may independently be any integer from 0-10 inclusive, or any integer therebetween;

R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand, and;

wherein one or more than one of V, S, R and W comprises one or more than one locked nucleic acid (LNA) monomer.

Single stranded nucleic acid molecules, or single-stranded RNA (ssRNA) molecules according to various embodiments of the invention that comprise at least one LNA, are generally described by Formula IVd:

Rₖ₃-Qₚ-(Dₘ)-Zₙ-Rₖ₄ Formula IVd

Formula IVd represents a single-stranded nucleic acid molecule having a configuration Rₖ₃-Qₚ-(Dₘ)-Zₙ-Rₖ₄ represented in a 5′-3′ direction (left to right) where:

n is any integer from 0 to 10, or any amount therebetween, with the proviso that if n=0, p=1, 2, 3, 4, 5, 6, 7, 8, 9 or 10;

p is any integer from 0 to 10, or any amount therebetween, with the proviso that if p=0, n=1, 2, 3, 4, 5, 6, 7, 8, 9 or 10;

Z and Q is any nucleoside, ribonucleoside, deoxyribonucleoside, nucleoside analogue, ribonucleoside analogue or deoxyribonucleoside analogue;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

D is cytosine, a cytosine-analogue nucleoside, uracil, or a uracil-analogue nucleoside;

k₃, and k₄ may independently be any integer from 0-10 inclusive, or any integer therebetween;

R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand, and;

wherein one or more than one of R, Z, D, and Q, comprises one or more than one locked nucleic acid (LNA) monomer.

In some embodiments of the invention, compositions may comprise single-stranded RNA molecules according to Formula IVc or Formula IVd, or both Formula IVc and Formula IVd in various molar ratios. For example, in some embodiments, single stranded RNA molecules according to Formula IVc and Formula IVd may be combined in about equimolar ratios. Some, none or all single-stranded RNA molecules according to Formula IVc and Formula IVd may hybridize with another complementary single-stranded RNA molecule to form double-stranded RNA molecules.

In other embodiments, single stranded RNA molecules according to Formula IVc may be combined in a composition with single stranded RNA molecules according to Formula IVd in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVc or Formula IVd may hybridize with another complementary single-stranded RNA molecule to form double-stranded RNA molecules.

In other embodiments, single stranded RNA molecules according to Formula IVd may be combined in a composition with single stranded RNA molecules according to Formula IVc in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVc or Formula IVd may hybridize with another complementary single-stranded RNA molecule to form double-stranded RNA molecules.

Non-limiting examples of single-stranded nucleic acids of the present invention may be as shown in any one of Formula IVe, IVf, IVg, IVh, IVi, or IVj, (shown in a 5′-3′ orientation, left to right), where I is a 2′-O-methyl-inosine nucleoside, C is a 2′-O-methyl-cytosine nucleoside, G is a 2′-O-methyl-guanosine nucleoside, T is a 2‘-O’methyl-thymidine nucleoside, A is a 2′-O-methyl-adenosine nucleoside, U is a 2′-O-methyl-uridine nucleoside, T_{LNA} is an thymidine nucleoside with an LNA ribose, G_{LNA} is a guanosine nucleoside with an LNA ribose, C_{LNA} is a cytosine nucleoside with an LNA ribose, A_{LNA} is an adenosine nucleoside with an LNA ribose, and m is 15:

(I₁₅)-G-T_{LNA}-G_{LNA}-A-T_{LNA}-A-T_{LNA}-G_{LNA} Formula IVe

(C₁₅)—C_{LNA}-A-T_{LNA}-A-T_{LNA}-C-A_{LNA}-C_{LNA} Formula IVf

G_{LNA}-(15)-G-T_{LNA}-G_{LNA}-A-T_{LNA}-A-T_{LNA} Formula IVg

C_{LNA}—(C₁₅)—C_{LNA}-A-T_{LNA}-A-U—C_{LNA}-A_{LNA} Formula IVh

T_{LNA}-G_{LNA}-(115)-T_{LNA}-T_{LNA}-A-T_{LNA}-A_{LNA} Formula IVi

A_{LNA}-C_{LNA}—(C₁₅)—C_{LNA}-A-T_{LNA}-A-T_{LNA}-C_{LNA} Formula IVj

In some embodiments of the invention, compositions may comprise single-stranded RNA molecules according to Formula IVe or Formula IVf, or both Formula IVe and Formula IVf in various molar ratios. For example, in some embodiments, single stranded RNA molecules according to Formula IVe and Formula IVf may be combined in about equimolar ratios. Some, none or all single-stranded RNA molecules according to Formula IVe and Formula IVf may hybridize with another complementary single-stranded RNA molecule to form double-stranded RNA molecules.

In other embodiments, single stranded RNA molecules according to Formula IVe may be combined in a composition with single stranded RNA molecules according to Formula IVf in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVe or Formula IVf may hybridize with another complementary single-stranded RNA molecule to form double-stranded RNA molecules.

In other embodiments, single stranded RNA molecules according to Formula IVf may be combined in a composition with single stranded RNA molecules according to Formula IVe in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVe or Formula IVf may hybridize with another complementary single-stranded RNA molecule to form double-stranded RNA molecules.

In some embodiments of the invention, compositions may comprise single-stranded RNA molecules according to Formula IVg or Formula IVh, or both Formula IVg and Formula IVh in various molar ratios. For example, in some embodiments, single stranded RNA molecules according to Formula IVg and Formula IVh may be combined in about equimolar ratios. Some, none or all single-stranded RNA molecules according to Formula IVg and Formula IVh may hybridize with another complementary single-stranded RNA molecule to form double-stranded RNA molecules.

In other embodiments, single stranded RNA molecules according to Formula IVg may be combined in a composition with single stranded RNA molecules according to Formula IVh in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVg or Formula IVh may hybridize with another complementary single-stranded RNA molecule to form double-stranded RNA molecules.

In other embodiments, single stranded RNA molecules according to Formula IVh may be combined in a composition with single stranded RNA molecules according to Formula IVg in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVg or Formula IVh may hybridize with another complementary single-stranded RNA molecule to form double-stranded RNA molecules.

In some embodiments of the invention, compositions may comprise single-stranded RNA molecules according to Formula IVi or Formula IVj, or both Formula IVi and Formula IVj in various molar ratios. For example, in some embodiments, single stranded RNA molecules according to Formula IVi and Formula IVj may be combined in about equimolar ratios. Some, none or all single-stranded RNA molecules according to Formula IVi and Formula IVj may hybridize with another complementary single-stranded RNA molecule to form double-stranded RNA molecules.

In other embodiments, single stranded RNA molecules according to Formula IVi may be combined in a composition with single stranded RNA molecules according to Formula IVj in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVi or Formula IVj may hybridize with another complementary single-stranded RNA molecule to form double-stranded RNA molecules.

In other embodiments, single stranded RNA molecules according to Formula IVj may be combined in a composition with single stranded RNA molecules according to Formula IVi in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVi or Formula IVj may hybridize with another complementary single-stranded RNA molecule to form double-stranded RNA molecules.

In some embodiments, pairs of single stranded nucleic acids, for example, Formulas IVe and IVf, or Formulas IVg and IVh, or Formulas IVi and IVj, may hybridize and/or concatemerize under some thermodynamic, ionic or pH conditions.

### Nucleic Acids Comprising CPG Motifs

Double-stranded nucleic acid molecule according to various embodiments of the invention that comprise a CpG motif, where the CpG motif comprises at least one LNA, are generally described by Formulas VIa-VId:

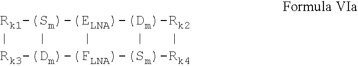

Formula VIa represents a double-stranded nucleic acid molecule having a first strand Rₖ₁—(Sₘ)-(E_{LNA})-(Dₘ)-Rₖ₂ and a second strand Rₖ₃-(Dₘ)-(F_{LNA})—(Sₘ)—Rₖ₄, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

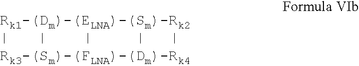

Formula VIb represents a double-stranded nucleic acid molecule having a first strand Rₖ₁-(Dₘ)-(E_{LNA})-(Sₘ)—Rₖ₂ and a second strand Rₖ₃—(Sₘ)—(F_{LNA})-(Dₘ)-Rₖ₄, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

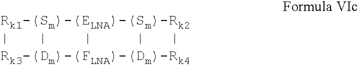

Formula VIc represents a double-stranded nucleic acid molecule having a first strand Rₖ₁—(Sₘ)-(E_{LNA})-(Sₘ)—Rₖ₂ and a second strand Rₖ₃-(Dₘ)-(F_{LNA})-(Dₘ)-Rₖ₄, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

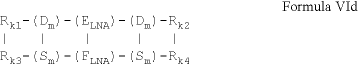

Formula VId represents a double-stranded nucleic acid molecule having a first strand Rₖ₁-(Dₘ)-(E_{LNA})-(Dₘ)-Rₖ₂ and a second strand Rₖ₃—(Sₘ)—(F_{LNA})—(Sₘ)—Rₖ₄, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

For each of Formula VIa-d;

E_{LNA} is CpG or a CpG motif, where one or more than one of the nucleosides, C, G, comprising the CpG or the CpG motif, is an LNA;

F_{LNA} is CpG or a CpG motif, where one or more than one of the nucleosides, C, G, comprising the CpG or the CpG motif, is an LNA;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage

In some embodiments of the invention which are not to be considered limiting in any manner, the CpG motif may comprise two hexamer sequences of LNA nucleosides:

(SEQ ID NO: 23)

E_{LNA} = 5′ - G_{LNA} T_{LNA} C_{LNA} G_{LNA} T_{LNA} T_{LNA} - 3′;
and

(SEQ ID NO: 24)

F_{LNA} = 5′ - A_{LNA} A_{LNA} C_{LNA} G_{LNA} A_{LNA} C_{LNA} - 3′.

Non-limiting examples of such sequences are generally described by Formulas VIe to VIh:

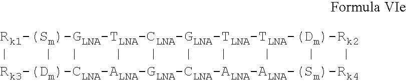

Formula VIe represents a double-stranded nucleic acid molecule having a first strand

Rₖ₁—(Sₘ)-G_{LNA}-T_{LNA}-C_{LNA}-G_{LNA}-T_{LNA}-T_{LNA}-(Dₘ)-Rₖ₂ VIi

and a second strand

Rₖ₃-(Dₘ)-C_{LNA}-A_{LNA}-G_{LNA}-C_{LNA}-A_{LNA}-A_{LNA}-(Sₘ)—Rₖ₄ VIj

with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

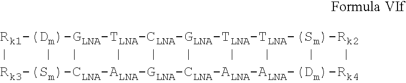

Formula VIf represents a double-stranded nucleic acid molecule having a first strand

Rₖ₁-(Dₘ)-G_{LNA}-T_{LNA}-C_{LNA}-G_{LNA}-T_{LNA}-T_{LNA}-(Sₘ)—Rₖ₂ VIk

and a second strand

Rₖ₃—(Sₘ)—C_{LNA}-A_{LNA}-G_{LNA}-C_{LNA}-A_{LNA}-A_{LNA}-(Dₘ)-Rₖ₄ VIl

with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

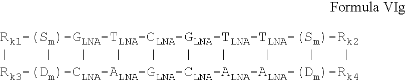

Formula VIg represents a double-stranded nucleic acid molecule having a first strand

Rₖ₁—(Sₘ)-G_{LNA}-T_{LNA}-C_{LNA}-G_{LNA}-T_{LNA}-T_{LNA}-(Sₘ)—Rₖ₂ VIm

and a second strand

Rₖ₃-(Dₘ)-C_{LNA}-A_{LNA}-G_{LNA}-C_{LNA}-A_{LNA}-A_{LNA}-(Dₘ)-Rₖ₄ VIn

with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

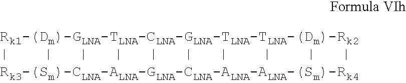

Formula VIh represents a double-stranded nucleic acid molecule having a first strand

Rₖ₁-(Dₘ)-G_{LNA}-T_{LNA}-C_{LNA}-G_{LNA}-T_{LNA}-T_{LNA}-(Dₘ)-Rₖ₂ VIo

and a second strand

Rₖ₃—(Sₘ)—C_{LNA}-A_{LNA}-G_{LNA}-C_{LNA}-A_{LNA}-A_{LNA}-(Sₘ)—Rₖ₄ VIp

with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

For each of Formulas VI e-VIh and VIi to VIp;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage

### Concatemeric Combinations

In some embodiments of the invention, the double-stranded nucleic acids comprising at least one CpG motif comprising at least one LNA nucleoside may include unpaired nucleosides, forming a ‘sticky end’ and may form concatemers. Formulae VIIa-VIIh (shown below in a 5′-3′ orientation, read left to right) represent single-stranded nucleic acids that hybridize according to sequence complementarity to form the double-stranded nucleic acids, for example as those described above in Formulas VIa to VIh. A double-stranded nucleic acid comprising a ‘sticky end’ may also be referred to as a monomer of a concatemeric polymer, according to some embodiments of the invention. Formula VIIa to VIIh are shown below followed by examples of combinations of nucleic acids comprising Formula VIIa to VIIh.

Rₖ₁—(Sₘ)-(E_{LNA}) Formula VIIa

Rₖ₂-(Dₘ)-(F_{LNA}) Formula VIIb

Rₖ₃-(Dₘ)-(E_{LNA}) Formula VIIc

Rₖ₄—(Sₘ)—(F_{LNA}) Formula VIId

(E_{LNA})-(Sₘ)—Rₖ₁ Formula VIIe

(F_{LNA})-(Dₘ)-Rₖ₂ Formula VIIf

(E_{LNA})-(Dₘ)-Rₖ₂ Formula VIIg

(F_{LNA})—(Sₘ)—Rₖ₂ Formula VIIh

For each of Formula VIIa-VIIh;

E_{LNA} is CpG or a CpG motif, where one or more than one of the nucleosides, C, G, comprising the CpG or the CpG motif is an LNA;

F_{LNA} is CpG or a CpG motif, where one or more than one of the nucleosides, C, G, comprising the CpG or the CpG motif is an LNA;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage

In some embodiments of the invention, compositions may comprise single-stranded RNA molecules according to one or more than one nucleic acid of Formula VIIa to VIIh, or a combination of at least two or more than two nucleic acids of Formula VIIa to VIIh in various molar ratios. For example, in some embodiments, single stranded RNA molecules according to Formula VIIa and Formula VIIb may be combined in about equimolar ratios. Some, none or all single-stranded RNA molecules according to Formula VIIc, Formula VIId, Formula VIIe, Formula VIIf, Formula VIIg, or Formula VIIh may hybridize with another complementary single-stranded RNA molecule to form double-stranded RNA molecules.

In other embodiments, single stranded RNA molecules according to Formula VIIa may be combined in a composition with single stranded RNA molecules according to Formula VIIb in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIa or Formula VIIb may hybridize with another complementary single-stranded RNA molecule to form double-stranded RNA molecules.

In other embodiments, single stranded RNA molecules according to Formula VIIc may be combined in a composition with single stranded RNA molecules according to Formula VIId in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIc or Formula VIId may hybridize with another complementary single-stranded RNA molecule to form double-stranded RNA molecules.

In other embodiments, single stranded RNA molecules according to Formula VIIe may be combined in a composition with single stranded RNA molecules according to Formula VIIf in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIe or Formula VIIf may hybridize with another complementary single-stranded RNA molecule to form double-stranded RNA molecules.

In other embodiments, single stranded RNA molecules according to Formula VIIg may be combined in a composition with single stranded RNA molecules according to Formula VIIh in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIg or Formula VIIh may hybridize with another complementary single-stranded RNA molecule to form double-stranded RNA molecules.

In other embodiments, single stranded RNA molecules according to Formula VIIg may be combined in a composition with single stranded RNA molecules according to Formula VIId in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIg or Formula VIId may hybridize with another complementary single-stranded RNA molecule to form double-stranded RNA molecules.

In other embodiments, single stranded RNA molecules according to Formula VIIa may be combined in a composition with single stranded RNA molecules according to Formula VIIf in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIa or Formula VIIf may hybridize with another complementary single-stranded RNA molecule to form double-stranded RNA molecules.

In other embodiments, single stranded RNA molecules according to Formula VIIe may be combined in a composition with single stranded RNA molecules according to Formula VIIb in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIe or Formula VIIb may hybridize with another complementary single-stranded RNA molecule to form double-stranded RNA molecules.

In other embodiments, single stranded RNA molecules according to Formula VIIc may be combined in a composition with single stranded RNA molecules according to Formula VIIh in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIc or Formula VIIh may hybridize with another complementary single-stranded RNA molecule to form double-stranded RNA molecules.

Exemplary base-pairing arrangements are illustrated below. Other pairings and arrangements of double-stranded nucleic acids according to various embodiments of the invention, will be apparent to those of skill in the art. For each exemplary pairing illustrated below, the first strand is provided in a 5′-3′ orientation, and the second strand is provided in a 3′-5′ orientation when read left to right, according to convention in the art.

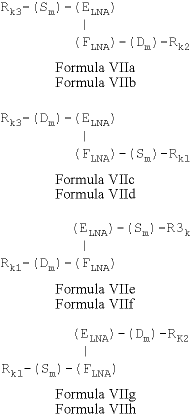

Alternate Pairings for Formulae VIIa-VIIh, Where k_{1, 2, 3, 4}=0:

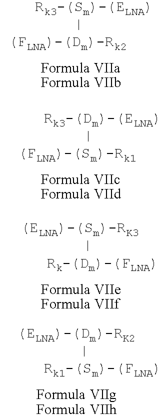

Such monomers may concatenate to form a longer or circular double-stranded nucleic acid polymer.

In some embodiments of the invention, the single-stranded nucleic acid molecules according to formulae VIIa-h may base-pair to form blunt-ended double-stranded nucleic acid molecules. Exemplary base-pairing arrangements are illustrated below.

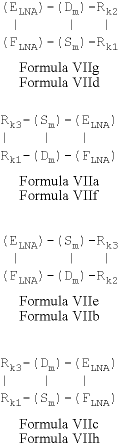

In the above example, k_{1, 2, 3, 4} is an integer from 1 to 10 (and not zero), R may be any nucleoside or group of nucleosides as described above, wherein at least one nucleoside from each of the first and second strands form a hydrogen-bonded base pairing.

In some embodiments, pairs of single stranded nucleic acids, for example Formula VIIa and VIIb, or Formula VIIc and VIId, or Formula VIIe and VIIf, or Formula VIIg and VIIh, or Formula VIIg and VIId, or Formula VIIa and VIIf, or Formula VIIe and VIIb, or Formula VIIc and VIIh, may concatemerize under some thermodynamic, ionic or pH conditions.

In some embodiments of the invention, the double-stranded nucleic acids comprising at least one CpG motif comprising at least one LNA nucleoside may include unpaired nucleosides, forming a ‘sticky end’ and may form concatemers. Formulae VIIIa-VIIIh (shown below in a 5′-3′ orientation, read left to right) represent single-stranded nucleic acids that hybridize according to sequence complementarity to form the double-stranded nucleic acids, for example as those described above in Formulas VIa to VIh, as those described above for Formulas VIIIa to VIIh. A double-stranded nucleic acid comprising a ‘sticky end’ may also be referred to as a monomer of a concatemeric polymer, according to some embodiments of the invention. Formula VIIIa to VIIIh are shown below followed by examples of combinations of nucleic acids comprising Formula VIIIa to VIIIh.

(Sₘ)—Rₖ₁-(E_{LNA}) Formula VIIIa

(Dₘ)-Rₖ₂—(F_{LNA}) Formula VIIIb

(Dₘ)-Rₖ₃-(E_{LNA}) Formula VIIIc

(Sₘ)—Rₖ₄—(F_{LNA}) Formula VIIId

(E_{LNA})-Rₖ₁—(Sₘ) Formula VIIIe

(F_{LNA})—Rₖ₂(Dₘ) Formula VIIIf

(E_{LNA})-Rₖ₂-(Dₘ) Formula VIIIg

(F_{LNA})—Rₖ₂—(Sₘ) Formula VIIIh

For each of Formula VIIIa-VIIIh;

E_{LNA} is CpG or a CpG motif, where one or more than one of the nucleosides, C, G, comprising the CpG or the CpG motif is an LNA;

F_{LNA} is CpG or a CpG motif, where one or more than one of the nucleosides, C, G, comprising the CpG or the CpG motif is an LNA;

m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;

S is inosine, an inosine-analogue nucleoside, adenine or an adenine-analogue nucleoside;

D is cytosine, a cytosine-analogue nucleoside, uracil, or a uracil-analogue nucleoside;

k₁, k₂, k₃, and k₄ may independently be any integer from 0-10 inclusive, or any integer therebetween;

R may independently be any ribonucleoside connected by an internucleoside linkage

In some embodiments of the invention, compositions may comprise single-stranded RNA molecules according to one or more than one nucleic acid of Formula VIIIa to VIIIh, or a combination of at least two or more than two nucleic acids of Formula VIIIa to VIIIh in various molar ratios. For example, in some embodiments, single stranded RNA molecules according to Formula VIIIa and Formula VIIb may be combined in about equimolar ratios. Some, none or all single-stranded RNA molecules according to Formula VIIIc, Formula VIIId, Formula VIIIe, Formula VIIIf, Formula VIIIg, or Formula VIIIh may hybridize with another complementary single-stranded RNA molecule to form double-stranded RNA molecules.

In other embodiments, single stranded RNA molecules according to Formula VIIIa may be combined in a composition with single stranded RNA molecules according to Formula VIIIb in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIIa or Formula VIIIb may hybridize with another complementary single-stranded RNA molecule to form double-stranded RNA molecules.

In other embodiments, single stranded RNA molecules according to Formula VIIIc may be combined in a composition with single stranded RNA molecules according to Formula VIIId in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIIc or Formula VIIId may hybridize with another complementary single-stranded RNA molecule to form double-stranded RNA molecules.

In other embodiments, single stranded RNA molecules according to Formula VIIIe may be combined in a composition with single stranded RNA molecules according to Formula VIIIf in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIIe or Formula VIIIf may hybridize with another complementary single-stranded RNA molecule to form double-stranded RNA molecules.

In other embodiments, single stranded RNA molecules according to Formula VIIIg may be combined in a composition with single stranded RNA molecules according to Formula VIIIh in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIIg or Formula VIIIh may hybridize with another complementary single-stranded RNA molecule to form double-stranded RNA molecules.

In other embodiments, single stranded RNA molecules according to Formula VIIIg may be combined in a composition with single stranded RNA molecules according to Formula VIIId in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIIg or Formula VIIId may hybridize with another complementary single-stranded RNA molecule to form double-stranded RNA molecules.

In other embodiments, single stranded RNA molecules according to Formula VIIIa may be combined in a composition with single stranded RNA molecules according to Formula VIIIf in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIIa or Formula VIIIf may hybridize with another complementary single-stranded RNA molecule to form double-stranded RNA molecules.

In other embodiments, single stranded RNA molecules according to Formula VIIIe may be combined in a composition with single stranded RNA molecules according to Formula VIIb in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIIe or Formula VIIIb may hybridize with another complementary single-stranded RNA molecule to form double-stranded RNA molecules.

In other embodiments, single stranded RNA molecules according to Formula VIIIc may be combined in a composition with single stranded RNA molecules according to Formula VIIIh in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIIc or Formula VIIIh may hybridize with another complementary single-stranded RNA molecule to form double-stranded RNA molecules.

Exemplary base-pairing arrangements are illustrated below. Other pairings and arrangements of double-stranded nucleic acids according to various embodiments of the invention, will be apparent to those of skill in the art. For each exemplary pairing illustrated below, the first strand is provided in a 5′-3′ orientation, and the second strand is provided in a 3′-5′ orientation when read left to right, according to convention in the art.

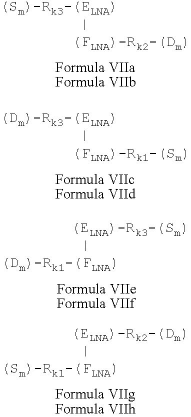

### Alternate Pairings for Formulae VIIIa-VIIIh:

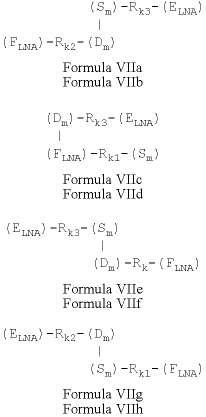

such monomers may concatenate to form a longer or circular double-stranded nucleic acid polymer.

In some embodiments of the invention, the single-stranded nucleic acid molecules according to formulae VIIIa-h may base-pair to form blunt-ended double-stranded nucleic acid molecules. Exemplary base-pairing arrangements are illustrated below.

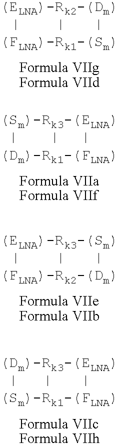

In the above examples, k_{1, 2, 3, 4} may be an integer from 0 to 10, R may be any nucleoside or group of nucleosides as described above. In some embodiments, where k is greater than zero, at least one nucleoside of R from each of the first and second strands forms a hydrogen-bonded base pairing.

In some embodiments, pairs of single stranded nucleic acids, for example Formula VIIIa and VIIIb, or Formula VIIIc and VIIId, or Formula VIIIe and VIIIf, or Formula VIIIg and VIIIh, or Formula VIII g and VIIId, or Formula VIIIa and VIIIf, or Formula VIIIe and VIIIb, or Formula VIIIc and VIIIh, may concatemerize under some thermodynamic, ionic or pH conditions.

Adjuvants or adjuvant compositions, according to various embodiments of the invention, comprise one or more than one nucleic acid species as described herein. The nucleic acid species may be single or double stranded. A combination of single and double stranded species may be present in an adjuvant or adjuvant composition.

The adjuvant or adjuvant composition may be a selective agonist for TLR3 or TLR9. In some embodiments of the invention, the adjuvant or adjuvant composition is an agonist for both TLR3 and TLR9. Examples of double-stranded nucleic acids comprising both TLR3 and TLR9 include those comprising two or more of Formulae VIIIa-h or Formulae VIIIa-h, or Formulae VIIIa-h and Formulae VIIIa-h.

Double-stranded nucleic acids according to some embodiments of the invention, for example those comprising two or more of Formulae VIIIa-h or Formula VIIIa-h, may be included in an adjuvant or adjuvant composition, to provide an adjuvant or adjuvant composition comprising both TLR3 and TLR9 agonist activity. The TLR3 and TLR9 agonist activity may be provided by a single species of double-stranded nucleic acid.

An IP-10 assay may be used to assess the ability of an adjuvant composition to provide TLR-3 agonist activity. Human HT29 cells secrete IP-10 into the culture supernatant as a result of stimulation with a TLR-3 agonist. IP-10 in the culture supernatant may be quantified, by, for example, ELISA. As another example, peripheral blood mononuclear cells (PBMCs) secrete cytokines into the supernatant as a result of stimulation with a TLR-3 agonist. The secreted cytokines, for example interferon-alpha,-beta and/or -gamma may be quantified by, for example ELISA. As another example, the maturation of immune effector cells, such as dendritic cells, may be assessed.

In vitro assays may be used to assess the ability of an adjuvant composition to provide TLR9 agonist activity. For example, the activity of a double-stranded nucleic acid composition may be assessed by B-cell proliferation assays or cytokine production by macrophages or dendritic cells. Examples of such assays are described in, for example, Jiang W et al 2006. Methods Mol Med 127:55-70.

Compositions according to various embodiments of the invention, including adjuvant compositions, may be administered as a dose from about 0.1 ug/kg to about 20 mg/kg of nucleic acid (based on the mass of the subject), or any amount therebetween, for example from about 1 ug to about 2000 ug/ml of nucleic acid or any amount therebetween, about 10 ug to about 1000 ug of nucleic acid or any amount therebetween, or about 30 ug to about 1000 ug of nucleic acid or any amount therebetween. For example, a dose of about 0.1, 0.5, 1.0, 2.0, 5.0, 10.0 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, 5000, 10000, 20000 ug of nucleic acid, or any amount therebetween may be used.

An “effective amount” of an adjuvant as used herein refers to the amount of adjuvant required to have an immunostimulatory effect when co-administered with an immunogen wherein the immunogen demonstrates biological activity. An immunogen may be present at an amount from about 0.1 ug/ml to about 20 mg/ml, or any amount therebetween, or about 1 ug/ml to about 2000 ug/ml, or any amount therebetween. An adjuvant may be present in an amount from about 0.1 ug/ml to about 20 mg/ml, or any amount therebetween, or about 1 ug/ml to about 2000 ug/ml, or any amount therebetween. The immunogen may be a killed whole-organism, a protein, a peptide, a fusion protein, a fusion peptide, a recombinant protein or a recombinant peptide. The immunogen may be HspE7.

Adjuvants according to various embodiments of the invention may be formulated with any of a variety of pharmaceutically acceptable excipients, frequently in an aqueous vehicle such as Water for Injection, Ringer's lactate, isotonic saline or the like. Pharmaceutically acceptable excipients include, for example, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. (1977. J. Pharm Sci. 66:1-19), or Remington—The Science and Practice of Pharmacy, 21^{st} edition. Gennaro et al editors. Lippincott Williams & Wilkins Philadelphia (both of which are herein incorporated by reference).

The excipients may also be carboxymethylcellulose or a polycationic polymer. Examples of polycationic polymers include but are not limited to poly-L-lysine, polyarginine, polyomithine, or a polypeptide comprising a majority of cationic amino acids. Molecular weight, concentrations and methods of preparation of such excipients may be found in, for example, U.S. Pat. No. 4,349,538 (which is incorporated herein by reference).

Compositions comprising an adjuvant according to various embodiments of the invention may be administered by any of several routes, including, for example, subcutaneous injection, intraperitoneal injection, intramuscular injection, intravenous injection, epidermal or transdermal administration, mucosal membrane administration, orally, nasally, rectally, or vaginally. See, for example, Remington—The Science and Practice of Pharmacy, 21^{st} edition. Gennaro et al editors. Lippincott Williams & Wilkins Philadelphia. Carrier formulations may be selected or modified according to the route of administration.

Compositions according to various embodiments of the invention may be provided in a unit dosage form, or in a bulk form suitable for formulation or dilution at the point of use.

Compositions according to various embodiments of the invention may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11^{th} edition. 2006. L L Brunton, editor. McGraw-Hill, New York, or Remington—The Science and Practice of Pharmacy, 21^{st} edition. Gennaro et al editors. Lippincott Williams & Wilkins Philadelphia.

In the context of the present invention, the terms “treatment”, “treating”, “therapeutic use,” or “treatment regimen” as used herein may be used interchangeably are meant to encompass prophylactic, palliative, and therapeutic modalities of administration of the compositions of the present invention, and include any and all uses of the presently claimed compounds that remedy a disease state, condition, symptom, sign, or disorder caused by an inflammation-based pathology, cancer, infectious disease, allergic response, hyperimmune response, or other disease or disorder to be treated, or which prevents, hinders, retards, or reverses the progression of symptoms, signs, conditions, or disorders associated therewith. Thus, any prevention, amelioration, alleviation, reversal, or complete elimination of an undesirable disease state, symptom, condition, sign, or disorder associated with an inflammation-based pathology, or other disease or disorder that benefits from stimulation of the body's immune response, is encompassed by the present invention. A treatment may comprise administration of an effective amount of a composition as described herein, alone or in combination with an immunogen.

Compositions according to various embodiments of the invention may further comprise one or more than one immunogen, for example a viral or bacterial (“pathogen”) immunogen. An immunogen may be prepared from a killed whole-organism (a ‘killed vaccine’) or may be prepared from a specific protein, peptide or other substructure of the pathogen. Alternatively, the immunogen may be a fusion protein comprising a whole or partial protein or peptide from a pathogen, fused with another non-pathogen protein or peptide, such as a “His-Tag” or other moiety useful in purification of the immunogen. Specific proteins or peptides may be produced using molecular biology techniques or methods (“recombinant” proteins or peptides). Conventional techniques or methods used in recombinant molecular biology are described in, for example, Molecular Cloning: a Laboratory Manual 3^{rd} edition. Sambrook and Russell. CSHL Press, Cold Spring Harbour, New York; Current Protocols in Molecular Biology, 2007 Ausubel et al editors. Wiley InterScience, New York; Current Protocols in Immunology, 2006 Coligan et al editors. Wiley InterScience, New York.

Examples of immunogens include, but are not limited to proteins comprising heat shock proteins, antigens from bacterial, fungal or viral pathogens, or heat shock fusion proteins for example but not limited to HspE7 (WO 99/07860, U.S. Pat. No. 7,157,089, both of which are incorporated herein by reference) comprising antigens from bacterial or viral pathogens. Examples of bacterial, fungal or viral pathogens include, but are not limited to, causative agents of the following diseases: papilloma, genital warts, influenza, hepatitis A, hepatitis B, hepatitis C, hepatitis D, hepatitis E, hepatitis G, Cytomegalovirus, Epstein, Barr virus, AIDS, AIDS Related Complex, Chickenpox (Varicella), Common cold, Cytomegalovirus Infection, Colorado tick fever—Dengue fever, Ebola haemorrhagic fever—Hand, foot and mouth disease, Hepatitis, Herpes simplex, Herpes zoster, HPV, Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease, Yellow fever, Anthrax, Bacterial Meningitis, Botulism, Brucellosis, Campylobacteriosis, Cat Scratch Disease, Cholera, Diphtheria, Epidemic Typhus, Gonorrhea, Impetigo, Legionellosis, Leprosy (Hansen's Disease), Leptospirosis, Listeriosis, Lyme Disease, Melioidosis, MRSA infection, Nocardiosis, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Typhus, urinary tract infections, aspergillosis, basidiobolomycosis, candidiasis, cryptococcosis, coccidioidomycosis, dermatophytosis, ringworm, histoplasmosis, fungemia, paracoccidioidomycosis, pneumocystis pneumonia, and the like. Recombinant immunogens may be expressed using a recombinant expression system, for example bacterial, yeast, baculoviral, mammalian cell or plant expression system.

In some embodiments of the invention, compositions according to various embodiments of the invention may be used for the treatment of a disease or disorder associated with a bacterial or viral pathogen. A disease or disorder associated with a bacterial or viral pathogen includes, but is not limited to, an active or latent infection with a bacterial or viral pathogen, an autoimmune response developed in conjunction with, or following an active or latent infection with a bacterial or viral pathogen, a side effect developed in conjunction with, or following an active or latent infection with a bacterial or viral pathogen.

In some embodiments of the invention, an immunogen may be a tumor antigen, or an antigen found in association with a cancer.

The term “cancer” has many definitions. According to the American Cancer Society, cancer is a group of diseases characterized by uncontrolled growth (and sometimes spread) of abnormal cells. Although often referred to as a single condition, it actually consists of more than 200 different diseases. Cancerous growths can kill when such cells prevent normal function of vital organs, or spread throughout the body, damaging essential systems. The composition of the present invention may be used to treat susceptible neoplasms in an animal or subject in a method that comprises administering to the animal or subject in need thereof an effective amount of a compound or composition of the present invention.

Non-limiting examples of different types of cancers against which compounds of the present invention may be effective as therapeutic agents include: carcinomas, such as neoplasms of the central nervous system, including glioblastoma multiforme, astrocytoma, oligodendroglial tumors, ependymal and choroid plexus tumors, pineal tumors, neuronal tumors, medulloblastoma, schwannoma, meningioma, and meningeal sarcoma; neoplasms of the eye, including basal cell carcinoma, squamous cell carcinoma, melanoma, rhabdomyosarcoma, and retinoblastoma; neoplasms of the endocrine glands, including pituitary neoplasms, neoplasms of the thyroid, neoplasms of the adrenal cortex, neoplasms of the neuroendocrine system, neoplasms of the gastroenteropancreatic endocrine system, and neoplasms of the gonads; neoplasms of the head and neck, including head and neck cancer, neoplasms of the oral cavity, pharynx, and larynx, and odontogenic tumors; neoplasms of the thorax, including large cell lung carcinoma, small cell lung carcinoma, non-small cell lung carcinoma, malignant mesothelioma, thymomas, and primary germ cell tumors of the thorax; neoplasms of the alimentary canal, including neoplasms of the esophagus, stomach, liver, gallbladder, the exocrine pancreas, the small intestine, veriform appendix, and peritoneum, adenocarcinoma of the colon and rectum, and neoplasms of the anus; neoplasms of the genitourinary tract, including renal cell carcinoma, neoplasms of the renal pelvis, ureter, bladder, urethra, prostate, penis, testis; and female reproductive organs, including neoplasms of the vulva and vagina, cervix, adenocarcinoma of the uterine corpus, ovarian cancer, gynecologic sarcomas, and neoplasms of the breast; neoplasms of the skin, including basal cell carcinoma, squamous cell carcinoma, dermatofibrosarcoma, Merkel cell tumor, and malignant melanoma; neoplasms of the bone and soft tissue, including osteogenic sarcoma, malignant fibrous histiocytoma, chondrosarcoma, Ewing's sarcoma, primitive neuroectodermal tumor, and angiosarcoma; neoplasms of the hematopoietic system, including myelodysplastic sydromes, acute myeloid leukemia, chronic myeloid leukemia, acute lymphocytic leukemia, HTLV-1 and 5, T-cell leukemia/lymphoma, chronic lymphocytic leukemia, hairy cell leukemia, Hodgkin's disease, non-Hodgkin's lymphomas, and mast cell leukemia; and neoplasms of children, including acute lymphoblastic leukemia, acute myelocytic leukemias, neuroblastoma, bone tumors, rhabdomyosarcoma, lymphomas, renal tumors, and the like.

In some embodiments of the invention, an immunogen may be an allergen. An allergen is an agent that induces an allergic response in a subject, upon exposure to the allergen. Chronic inflammation observed in allergic and asthmatic disorders resulting from inhaled allergens is largely dominated by localized tissue infiltration of eosinophils, and hyperreactivity of the tissues to the allergen. Inflammation may be reduced through use of corticosteroids and/or bronchodilators, however these do not treat the root cause. As discussed in WO 99/07860, allergen-specific T-lymphocytes are selectively enriched in such hyperreactive tissue, and this sensitivity may be dependent on early antigen exposure in childhood or infancy.

Selection for specific Th1-versus Th2-like memory cells in an individual immune response to inhaled antigens occurs in the regional lymph nodes draining the conducting airways. This selections may be regulated by a variety of cytokines produced by antigen specific CD4+ and CD8+ T-cells. This T-cell selection process may be influenced by infectious agents: infections in the airway mucosa may mobilize and activate local tissue (alveolar) macrophages which migrate to the regional lymph nodes and secrete Th2 inhibitory cytokines such as IL-12 and alpha-interferon. In addition, they may add to the gamma-interferon levels in the milieu through activation of natural killer cells. The net result is the production of CTLs (which are predominantly CD8+ cells). Gamma-interferon inhibits the generation of Th2 cells and therefore production of IL-4 and IL-5, cytokines crucial for the generation of humoral (IgE) and cellular (eosinophils, basophils and mast cells) allergic responses (Anderson, G. P. and Coyle, A. J., Trends Pharmacol. Sci., 15:324-332 (1995); Stam, W. B., van Oosterhout, A. J. and Nijkamp, F. P., Life Sci., 53:1921-1934 (1993)).

In mammals, stress proteins have been shown to induce humoral as well as cellular immune responses. When a soluble antigen mixed with, chemically conjugated to or fused to a stress protein is administered to a mammal, cell-mediated cytolytic immune responses are substantially enhanced. These responses are largely due to CD8+ T cells. Therefore, a comparison of the CD4+ responses to antigens by themselves to those mixed with or coupled to stress proteins give the predicted profile: soluble antigens mixed with or linked to stress proteins yield a high proportion of CTLs (mainly CD8+ T cells) which are a measure of stimulation of the Th1 pathway described before because these CTLs arose as a result of the induction of antigen specific T cells of the Th1 type. These Th1 cells produce gamma-interferon, which inhibits Th2 cells. Therefore, the Th2 cytokines IL-4 and IL-5 are no longer available to support the production of IgE and eosinophils. With decreasing titer of IgE, direct antigenic stimulation of mast and basophil cells will decline. In addition, decreased IL-5 production will lead to decreased production, differentiation and activation of eosinophils. This pattern will cause decreased inflammation of the involved tissue and result in less hyperreactive (asthmatic) events.

Therefore, administration of mixtures of known allergenic antigens (allergens), or stress proteins or compositions comprising allergens chemically linked to or fused to stress proteins in combination with agents according to Formula II, IIa-e, Formula III, IIIa-d, Formula IVa-j, combinations of at least two of Formula IVa-j, Formula Va to Formula Vc, Formula VIa to Formula VIh, Formula VIj to Formula VIo, Formula VIIa to VIIh, Formula VIIIa to Formula VIIIH, in various molar ratios may influence the Th1 to Th2 ratio in atopic patients, restoring a more normal balance and leading to decreased allergic or asthmatic response.

Therefore, the invention provides for a TLR3 agonist, or a TLR9 agonist, or a composition that is both a TLR3 and a TLR9 agonist, and an adjuvant or adjuvant composition that comprises a TLR3 agonist, or a TLR9 agonist, or a composition that is both a TLR3 and a TLR9 agonist.

In some embodiments of the invention, the immunogen includes HspE7. Methods for producing the HspE7 fusion protein are described in WO 99/07860 and U.S. 60/803,606, both of which are herein incorporated by reference.

### Sequences

For the sequence according to SEQ ID NO: 1, residues G1, G2, G25 and G26 are LNA residues; residues 3 to 24 are inosine ribonucleotides.

For the sequence according to SEQ ID NO: 2, residues C1, C2, C25 and C26 are LNA residues; residues C3 to C24 are ribonucleotides.

For the sequence according to SEQ ID NO: 3, residues T17, G18, T20, T22 and G23 are LNA residues; residues 1 to 15 are inosine ribonucleotides; residues G16, A19 and A21 may be ribonucleotides or deoxyribonucleotides.

For the sequence according to SEQ ID NO: 4, residues C16, T18, T20, A22 and C23 are LNA residues; residues C1 to C15 are ribonucleotides; residues A17, A19 and C21 may be ribonucleotides or deoxyribonucleotides.

For the sequence according to SEQ ID NO: 5, residues G1, T18, T19, T21 and T23 are LNA residues; residues 1 to 17 are inosine ribonucleotides; residues G17, A20 and a22 may be ribonucleotides or deoxyribonucleotides.

For the sequence according to SEQ ID NO: 6, residues C1, C17, T19, C22 and C23 are LNA residues; residues C2 to C16 are ribonucleotides; residues A18, A20 and U21 may be ribonucleotides or deoxyribonucleotides.

For the sequence according to SEQ ID NO: 7, residues T1, T2, T18, T19, T21 and A22 are LNA residues residues 3 to 17 are inosine ribonucleotides; residues A19 and A21 may be ribonucleotides or deoxyribonucleotides.

For the sequence according to SEQ ID NO: 8, residues A1, C2, C18, T20, T22 and C23 are LNA residues C3 to C17 are ribonucleotides; residues A19 and A21 may be ribonucleotides or deoxyribonucleotides.

For the sequence according to SEQ ID NO: 9, residues G1 and G2 are LNA residues; residues 2 to 17 are inosine ribonucleotides and A18 to A32 are ribonucleotides.

For the sequence according to SEQ ID NO: 10, residues C16 and C17 are LNA residues; residues C1 to C15 and U18 to U32 are ribonucleotides.

For the sequence according to SEQ ID NO: 11, residues G1 and G2 are LNA residues; residues 3 to 12 are inosine ribonucleotides and A13 to A22 are ribonucleotides.

For the sequence according to SEQ ID NO: 12, residues C11 and C12 are LNA residues; residues U1 to U10 and C13 to C22 are ribonucleotides.

For the sequence according to SEQ ID NO: 13, residues G1, G2, G18, T19, C20, G21, T11, T23, G39 and G40 are LNA residues; residues 3 to 17 and 24 to 38 are inosine ribonucleotides.

For the sequence according to SEQ ID NO: 14, residues C1, C2, A18, A19, C20, G21, A22, C23, C39 and C40 and C23 are LNA residues; residues C3 to C17 and C24 to C38 are ribonucleotides.

For the sequence according to SEQ ID NO: 15, residues G1, G2, G18, T19, C20, G21, T22 and T23 are LNA residues; residues 3 to 17 are inosine ribonucleotides.

For the sequence according to SEQ ID NO: 16, residues A1, A2, C3, G4, A5, C6, C22 and C23 are LNA residues; residues C7 to C21 are ribonucleotides.

For the sequence according to SEQ ID NO: 17, residues G16, T17, C18, G19, T20, and T21 are LNA residues; residues 1 to 15 are inosine ribonucleotides.

For the sequence according to SEQ ID NO: 18, residues A16, A17, C18, G19, A20, and C21 are LNA residues; residues C1 to C15 are ribonucleotides.

For the sequence according to SEQ ID NO: 19, residues G1, G17, T18, C19, G20, T21 and T22 are LNA residues; residues 2 to 16 are inosine ribonucleotides.

For the sequence according to SEQ ID NO: 20, residues C1, A17, A18, C19, G20, A21 and C22 are LNA residues; residues C2 to C16 are ribonucleotides.

For the sequence according to SEQ ID NO: 21, residues C₁, G2, G18, T19, C20, G21, T22 and T23 are LNA residues; residues 3 to 17 are inosine ribonucleotides.

For the sequence according to SEQ ID NO: 22, residues G1, C2, A18, A19, C20, G21, A22 and C23 are LNA residues; residues C3 t C17 are ribonucleotides.

For the sequence according to SEQ ID NO: 23, all of the six residues are LNA residues.

For the sequence according to SEQ ID NO: 24, all of the six residues are LNA residues.

For the sequence according to SEQ ID NO: 25, residues C2 and C3 are LNA residues; residues C1 to C10 and U13 to U23 are ribonucleotides.

For the sequence according to SEQ ID NO: 26, residues G1 and G2 are locked nucleic acid residues; C3, G4, T5, C6, G7, T8, T9, A10, T26, G27, T28, C29, G30, T31, T32, G33 are deoxyribonucleotides; A11 to A25 inclusive are ribonucleotides.

For the sequence according to SEQ ID NO: 27, residues U1 to U15 inclusive are ribonucleotides; residues T16, A17 A18, C19, G20, A21, C22, G23, C26, A27, A28, C29, G30, A31, C32 and A33 are deoxyribonucleotides; C24 and C25 are locked nucleic acid residues.

The “LNA” subscript in combination with a single letter base designation (A, C, G, T, U, I) indicates that the associated nucleotide is a locked nucleic acid residue, comprising a 2′-4′ as described above.

### EXAMPLE 1

### Preparation of Double-Stranded Oligomers: GCLNA-polyIc-GCLNA

Oligomers according to SEQ ID NO: 1 and SEQ ID NO: 2 were synthesized using 2′-OMe-1-CE Phosphoramidites, 2′-OMe-C-CE Phosphoramidites, 5-Me-Bz-C-LNA-CE phosphoramidites and dmf-G-LNA-CE phosphoramidites according to standard techniques, as per manufacturer's protocols (Glen Research, Sterling Va.).

| | |
|---|---|
| G_{LNA}-G_{LNA}-(I₂₂)-G_{LNA}-G_{LNA} | (SEQ ID NO:1) |
| and | |
| C_{LNA}-C_{LNA}-(C₂₂)-C_{LNA}-C_{LNA}. | (SEQ ID NO:2) |

Equimolar amounts of each of the first and second oligomers were combined and permitted to anneal to produce the dsRNA compound GC_{LNA}-polylC-GCLNA, shown in Formula IIIa:

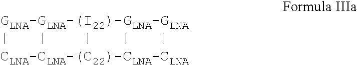

### EXAMPLE 2

### Preparation of Double-Stranded Oligomers with 3′ Unpaired Ends

Oligomers according to SEQ ID NO: 3, SEQ ID NO: 4, SEQ ID NO:5, SEQ ID NO: 6, SEQ ID NO: 7, and SEQ ID NO: 8 may be synthesized using 5-Me-Bz-C-LNA-CE Phosphoramidites, Bz-A-LNA-CE Phosphoramidites, dmf-G-LNA-CE Phosphoramidites, T-LNA-CE Phosphoramidites, 2′-OMe-1-CE Phosphoramidites, 2′-OMe-C-CE Phosphoramidites, 2′-OMe-A-CE Phosphoramidites, 2′-OMe-G-CE Phosphoramidites and 2′-OMe-U-CE Phosphoramidites according to standard techniques, as per manufacturer's protocols (Glen Research, Sterling Va.).

(SEQ ID NO: 3)

(I₁₅)-G-T_{LNA}-G_{LNA}-A-T_{LNA}-A-T_{LNA}-G_{LNA}

(SEQ ID NO: 4)

(C₁₅)-C_{LNA}-A-T_{LNA}-A-T_{LNA}-C-A_{LNA}-C_{LNA}

(SEQ ID NO: 5)

G_{LNA}-(I₁₅)-G-T_{LNA}-G_{LNA}-A-T_{LNA}-A-T_{LNA}

(SEQ ID NO: 6)

C_{LNA}-(C₁₅)-C_{LNA}-A-T_{LNA}-A-U-C_{LNA}-A_{LNA}

(SEQ ID NO: 7)

T_{LNA}-G_{LNA}-(I₁₅)-T_{LNA}-T_{LNA}-A-T_{LNA}-A_{LNA}

(SEQ ID NO: 8)

A_{LNA}-C_{LNA}-(C₁₅)-C_{LNA}-A-T_{LNA}-A-T_{LNA}-C_{LNA}

Equimolar amounts of each of SEQ ID NO: 3 and SEQ ID NO: 4, or SEQ ID NO:5 and SEQ ID NO: 6 or SEQ ID NO: 7 and SEQ ID NO: 8 may be combined and permitted to anneal to produce the double-stranded nucleic acid compounds shown in Formula Va, Vb and Vc, respectively.

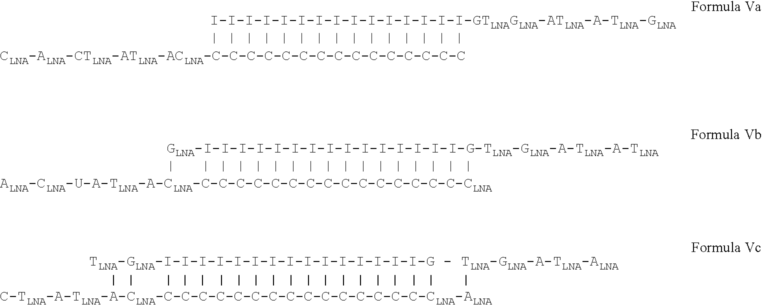

### EXAMPLE 3

### Preparation of Double-Stranded Oligomers with 3′ Unpaired Ends

Oligomers according to SEQ ID NO: 9, SEQ ID NO: 10, SEQ ID NO:11 and SEQ ID NO: 12 may be synthesized using 5-Me-Bz-C-LNA-CE Phosphoramidites, Bz-A-LNA-CE Phosphoramidites, dmf-G-LNA-CE Phosphoramidites, T-LNA-CE Phosphoramidites, 2′-OMe-1-CE Phosphoramidites, 2′-OMe-C-CE Phosphoramidites, 2′-OMe-A-CE Phosphoramidites, 2′-OMe-G-CE Phosphoramidites and 2′-OMe-U-CE Phosphoramidites according to standard techniques, as per manufacturer's protocols (Glen Research, Sterling Va.).

| | |
|---|---|
| G_{LNA}-G_{LNA}-(I)₁₅-(A)₁₅ | (SEQ ID NO: 9) |
| (C)₁₅-C_{LNA}-C_{LNA}-(U)₁₅ | (SEQ ID NO: 10) |
| G_{LNA}-G_{LNA}-(I)₁₀-(A)₁₀ | (SEQ ID NO: 11) |
| (U)₁₀-C_{LNA}-C_{LNA}-(C)₁₀ | (SEQ ID NO: 12) |
| (C)₁₀-C_{LNA}-C_{LNA}-(U)₁₀ | (SEQ ID NO: 25) |

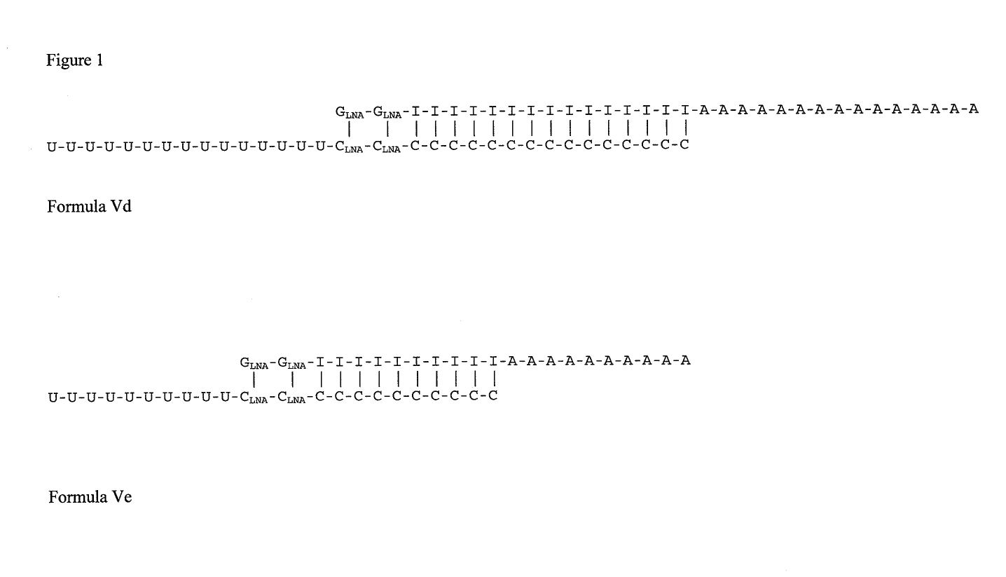

Equimolar amounts of each of SEQ ID NO: 9 and SEQ ID NO: 10, or SEQ ID NO: 11 and SEQ ID NO: 12, or SEQ ID NO: 11 and SEQ ID NO: 25, may be combined and permitted to anneal to produce the double-stranded nucleic acid compounds shown in Formula Vd and Ve, respectively (FIG. 1).

### EXAMPLE 4

### In Vitro Biological Activity of dsRNA in Combination with an Immunogen

A composition comprising HspE7, produced according to the method of U.S. 60/803,606 (which is incorporated herein by reference) and GC_{LNA}-polylC-GCLNA produced according to Example 1 above, may be tested for biological activity in vitro.

Augmentation of the ability of HspE7 to induce E7-specific CD8-positive T-lymphocytes (as an exemplary antiviral therapeutic approach) may be determined in the presence of GC_{LNA}-polylC-GCLNA. Naïve C57B1/6 mice may be injected subcutaneously, with either HspE7 alone, or HspE7 plus GC_{LNA}-polylC-GCLNA. After a time interval, for example 5 days, spleens may be removed from the mice and the number of E7-specific splenocytes measured by ELISPOT, for example, by using E7 specific class I MHC binding peptide E749-57 (RAHYNIVTF; Dalton Chemical Laboratories), or a control peptide HBCAg93-100 (MGLKFRQL; Dalton Chemical Laboratories) as recall antigens.

### EXAMPLE 5

### In Vivo Biological Activity of dSRNA in Combination with an Immunogen

A composition comprising HspE7, produced according to the method of PCT Publication WO 2007/137427 (which is incorporated herein by reference) and GC_{LNA}-polylC-GC_{LNA} produced according to Example 1 above, may be tested for biological activity in vivo.

In an exemplary method of a cancer therapeutic method, TC-1 tumors are first established in naïve C57B1/6 mice. Mice were injected in the flank with 6×1 TC-1 tumor cells. On day 7, mice bearing established TC-1 tumors may be injected subcutaneously in the scruff of the neck with either diluent, purified HspE7 alone, or graded doses of purified HspE7 mixed with different doses of GC_{LNA}-polylC-GCLNA. Mice are followed for tumor growth for an additional time interval, for example, 42 days—in this example, mice free of tumor 49 days post tumor implantation may be considered to be tumor free.

### EXAMPLE 6

### Preparation of Double-Stranded Oligomers Comprising CpG Motifs

Oligomers according to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21 and SEQ ID NO: 22 were synthesized using 2′-OMe-1-CE Phosphoramidites, 2′-OMe-C-CE Phosphoramidites, 5-Me-Bz-C-LNA-CE phosphoramidites and dmf-G-LNA-CE phosphoramidites according to standard techniques, as per manufacturer's protocols (Glen Research, Sterling Va.).

(SEQ ID NO: 13)

G_{LNA}-G_{LNA}-(I)₁₅-G_{LNA}-T_{LNA}-C_{LNA}-G_{LNA}-T_{LNA}-T_{LNA}-
(I)₁₅-G_{LNA}-G_{LNA}

(SEQ ID NO: 14)

C_{LNA}-C_{LNA}-(C)₁₅-A_{LNA}-A_{LNA}-C_{LNA}-G_{LNA}-A_{LNA}-C_{LNA}-
(C)₁₅-C_{LNA}-C_{LNA}

(SEQ ID NO: 15)

G_{LNA}-G_{LNA}-(I)₁₅-G_{LNA}-T_{LNA}-C_{LNA}-G_{LNA}-T_{LNA}-T_{LNA}-

(SEQ ID NO: 16)

A_{LNA}-A_{LNA}-C_{LNA}-G_{LNA}-A_{LNA}-C_{LNA}-(C)₁₅-C_{LNA}-C_{LNA}

(SEQ ID NO: 17)

(I)₁₅-G_{LNA}-T_{LNA}-C_{LNA}-G_{LNA}-T_{LNA}-T_{LNA}

(SEQ ID NO: 18)

(C)₁₅-A_{LNA}-A_{LNA}-C_{LNA}-G_{LNA}-A_{LNA}-C_{LNA}

(SEQ ID NO: 19)

G_{LNA}-(I)₁₅-G_{LNA}-T_{LNA}-C_{LNA}-G_{LNA}-T_{LNA}-T_{LNA}-

(SEQ ID NO: 20)

C_{LNA}-(C)₁₅-A_{LNA}-A_{LNA}-C_{LNA}-G_{LNA}-A_{LNA}-C_{LNA}

(SEQ ID NO: 21)

C_{LNA}-G_{LNA}-(I)₁₅-G_{LNA}-T_{LNA}-C_{LNA}-G_{LNA}-T_{LNA}-T_{LNA}

(SEQ ID NO: 22)

G_{LNA}-C_{LNA}-(C)₁₅-A_{LNA}-A_{LNA}-C_{LNA}-G_{LNA}-A_{LNA}-C_{LNA}

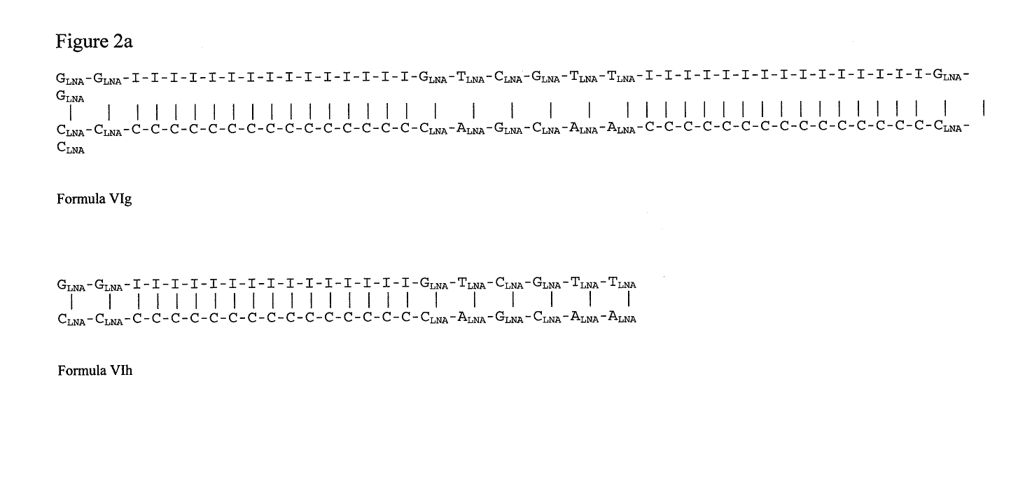

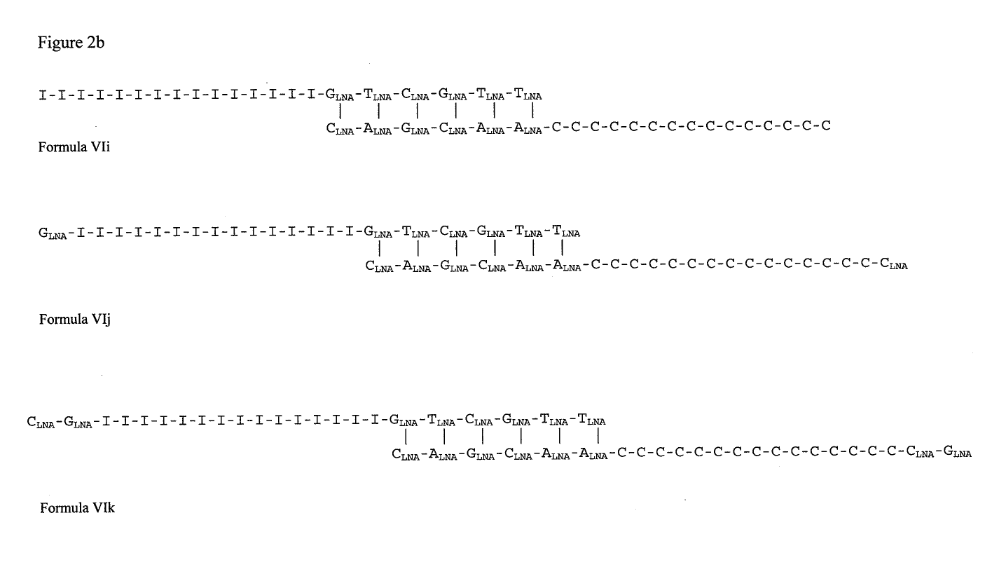

Equimolar amounts of each of SEQ ID NO: 13 and SEQ ID NO: 14, or SEQ ID NO: 15 and SEQ ID NO: 16, or SEQ ID NO: 17 and SEQ ID NO: 18, or SEQ ID NO: 19 and SEQ ID NO: 20, or SEQ ID NO: 21 and SEQ ID NO: 22 were combined and permitted to anneal to produce the dsRNA compound according to Formula VIg, VIh, VIi, VIj and VIk (FIG. 2).

### EXAMPLE 7

### Preparation of Double-Stranded Oligomers Comprising CpG and Poly a:U Motifs

Oligomers according to SEQ ID NO: 26 and SEQ ID NO: 27 were synthesized using 2′-OMe-A-CE Phosphoramidites, 2′-OMe-U-CE Phosphoramidites, DMT-dA-phosphoramidites, DMT-dC-phosphoramidites, DMT-dG-phosphoramidites, DMT-dT-phosphoramidites, 5-Me-Bz-C-LNA-CE phosphoramidites and dmf-G-LNA-CE phosphoramidites according to standard techniques, as per manufacturer's protocols (Eurogentec North America).

(SEQ ID NO: 26)

G_{LNA}-G_{LNA}-dC-dG-dT-dC-dG-dT-dT-dA-(rA)₁₅-dT-dG-dT-
dC-dG-dT-dT-dG

(SEQ ID NO: 27)

(rU)₁₅-dT-dA-dA-dC-dG-dA-dC-dG-C_{LNA}-C_{LNA}-dC-dA-dA-
dC-dG-dA-dC-dA

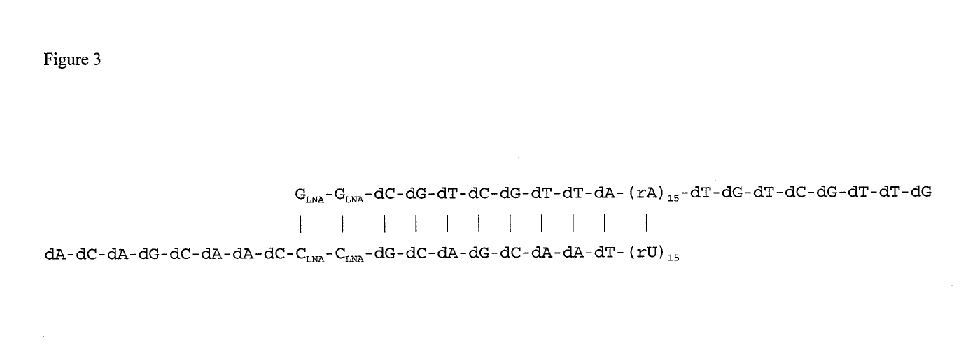

Equimolar amounts of each of SEQ ID NO: 26 and SEQ ID NO: 27 may be combined and permitted to anneal to produce the double-stranded nucleic acid compound shown in FIG. 3, having a section of unpaired nucleosides at either end. The unpaired nucleosides may allow concatemerization of the compound.

All citations are herein incorporated by reference.

One or more currently preferred embodiments have been described by way of example. It will be apparent to persons skilled in the art that a number of variations and modifications can be made without departing from the scope of the invention as defined in the claims.

What is claimed is:

1. A compound of the formula: 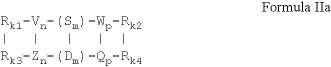
where:
n is any integer from 0 to 10, or any amount therebetween, with the proviso that if n=0, p=1, 2, 3, 4, 5, 6, 7, 8, 9 or 10;
p is any integer from 0 to 10, or any amount therebetween, with the proviso that if p=0, n=1, 2, 3, 4, 5, 6, 7, 8, 9 or 10;
V, Z, Q and W is any nucleoside, ribonucleoside, deoxyribonucleoside, nucleoside analogue, ribonucleoside analogue or deoxyribonucleoside analogue;
m is any integer from 1 to 500, or any amount therebetween;
S is inosine, an inosine-analogue nucleoside, adenine or an adenine-analogue nucleoside;
D is cytosine, a cytosine-analogue nucleoside, uracil, or a uracil-analogue nucleoside;
k₁, k₂, k₃, and k₄ may independently be any integer from 0-10 inclusive, or any integer therebetween;
R may independently be any ribonucleoside connected by an internucleoside linkage group to the geminal nucleoside, or R may be absent; and
wherein one or more than one of V, S, D, Z, Q, R and W comprises one or more than one locked nucleic acid (LNA) monomer.

2. A compound of the formula:
Rₖ₁—Vₙ—(Sₘ)—Wₚ—Rₖ₂ Formula IVc
where:
n is any integer from 0 to 10, or any amount therebetween, with the proviso that if n=0, p=1, 2, 3, 4, 5, 6, 7, 8, 9 or 10;
p is any integer from 0 to 10, or any amount therebetween, with the proviso that if p=0, n=1, 2, 3, 4, 5, 6, 7, 8, 9 or 10;
V and W is any nucleoside, ribonucleoside, deoxyribonucleoside, nucleoside analogue, ribonucleoside analogue or deoxyribonucleoside analogue;
m is any integer from 1 to 500, or any amount therebetween;
S is inosine, an inosine-analogue nucleoside, adenine or an adenine-analogue nucleoside;
k₁ and k₂ may independently be any integer from 0-10 inclusive, or any integer therebetween;
R may independently be any ribonucleoside connected by an internucleoside linkage group to the geminal nucleoside, or R may be absent; and
wherein one or more than one of V, S, R and W comprises one or more than one locked nucleic acid (LNA) monomer.

3. A compound of the formula
Rₖ₄-Qₚ-(Dₘ)-Zₙ-Rₖ₃ Formula IVd
where:
n is any integer from 0 to 10, or any amount therebetween, with the proviso that if n=0, p=1, 2, 3, 4, 5, 6, 7, 8, 9 or 10;
p is any integer from 0 to 10, or any amount therebetween, with the proviso that if p=0, n=1, 2, 3, 4, 5, 6, 7, 8, 9 or 10;
Z and Q is any nucleoside, ribonucleoside, deoxyribonucleoside, nucleoside analogue, ribonucleoside analogue or deoxyribonucleoside analogue;
m is any integer from 1 to 500, or any amount therebetween;
D is cytosine, a cytosine-analogue nucleoside, uracil, or a uracil-analogue nucleoside;
k₃ and k₄ may independently be any integer from 0-10 inclusive, or any integer therebetween;
R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand, and;
wherein one or more than one of R, Z, D, and Q, comprises one or more than one locked nucleic acid (LNA) monomer.

4. A method of making a compound of the formula 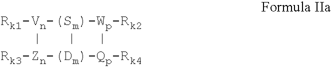
where
n is any integer from 0 to 10, or any amount therebetween, with the proviso that if n=0, p=1, 2, 3, 4, 5, 6, 7, 8, 9 or 10;
p is any integer from 0 to 10, or any amount therebetween, with the proviso that if p=0, n=1, 2, 3, 4, 5, 6, 7, 8, 9 or 10;
V, W, Z and Q is any nucleoside;
m is any integer from 1 to 500;
S is inosine, an inosine-analogue nucleoside, adenine or an adenine-analogue nucleoside;
D is cytosine, a cytosine-analogue nucleoside, uracil, or a uracil-analogue nucleoside;
k₁, k₂, k₃, and k₄ may independently be any integer from 0-10 inclusive, or any integer therebetween;
R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand, and;
wherein one or more than one of V, S, W, Z, D, and Q, comprises one or more than one LNA monomer, the method comprising:
mixing a molar ratio from about 0.5-1.0 to about 1.0-0.5 of an oligomer according to the compound of claim 2 with an oligomer according to the compound of claim 3, and annealing said first and second oligomers to form the double-stranded compound.

5. The compound of any of claims 1-3, wherein S is inosine and D is cytosine.

6. A composition comprising the compound of claim 5, poly-L-lysine and carboxymethylcellulose.

7. A composition comprising the compound of claim 5 and an immunogen.

8. A compound of the formula 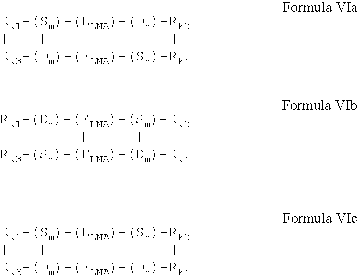
where
E_{LNA} is CpG or a CpG motif, where one or more than one of the nucleosides, C, G, comprising the CpG or the CpG motif is an LNA;
F_{LNA} is CpG or a CpG motif, where one or more than one of the nucleosides, C, G, comprising the CpG or the CpG motif is an LNA;
m is any integer from 1 to 500, or any amount therebetween;
S is inosine, an inosine-analogue nucleoside, adenine or an adenine-analogue nucleoside;
D is cytosine, a cytosine-analogue nucleoside, uracil, or a uracil-analogue nucleoside;
k₁, k₂, k₃, and k₄ may independently be any integer from 0-10 inclusive, or any integer therebetween;
R may independently be any ribonucleoside connected by an internucleoside linkage.

9. A compound according to formula VIa, VIb, VIc or VId of claim 8, wherein S is inosine and D is cytosine

10. A composition comprising the compound of claim 9 and an immunogen.

11. A compound comprising:
a first single-stranded nucleotide polymer comprising from one to 500 inosine, cytosine or a combination of inosine and cytosine ribonucleotides and from one to ten locked nucleic acid residues; and
a second single-stranded nucleotide polymer comprising from one to 500 inosine, cytosine or a combination of inosine and cytosine ribonucleotides and from one to ten locked nucleic acid residues;
where the first single-stranded nucleotide polymer and the second single-stranded nucleotide polymer are hydrogen bonded to form a double-stranded nucleic acid, the double-stranded nucleic acid comprising a double-stranded polylC region and a double-stranded region comprising locked nucleic acid residues.

12. A compound comprising:
a first single-stranded nucleotide polymer comprising from one to 500 inosine, cytosine or combination of inosine and cytosine ribonucleotides and a nucleic acid sequence according to SEQ ID NO: 23; and
a second single-stranded nucleotide polymer comprising from one to 500 inosine, cytosine or a combination of inosine and cytosine ribonucleotides and a nucleic acid sequence according to SEQ ID NO: 24;
where the first single-stranded nucleotide polymer and the second single-stranded nucleotide polymer are hydrogen bonded to form a double-stranded nucleic acid, the double-stranded nucleic acid comprising a double-stranded polylC region and a double-stranded region comprising locked nucleic acid residues.

13. An adjuvant composition comprising the compound of claim 11.

14. An adjuvant composition comprising the compound of claim 12

15. A TLR3 and TLR9 agonist comprising the compound of claim 12.
